(19) United States
(12) Patent Application Publication
(10) Pub. No.: US 2024/0390332 A1
(43) Pub. Date: Nov. 28, 2024

(54) Combination treatment and/or prevention of renal diseases and/or hypertension in non-human mammals comprising one or more SGLT-2 inhibitors and telmisartan

(71) Applicant: BOEHRINGER INGELHEIM VETMEDICA GMBH, Ingelheim am Rhein (DE)

(72) Inventors: Carla KROH, Oestrich-Winkel (DE); Silke HAAG-DIERGARTEN, IFrankfurt am Main (DE); Ingo Ulrich LANG, Ingelheim am Rhein (DE); Dagmar Maria POLOTZEK, Frankfurt am Main (DE); Tanja Margrit ZIMMERING, Ingelheim am Rhein (DE)

(21) Appl. No.: 18/666,922

(22) Filed: May 17, 2024

(30) Foreign Application Priority Data
May 24, 2023 (EP) 23175200.7

(51) Int. Cl. A61K 31/4184; A61K 9/00; A61K 9/08; A61K 31/70; A61K 31/7042; A61K 31/7048; A61K 31/7056; A61K 47/54; A61P 9/12; A61P 13/12

(52) U.S. Cl. CPC A61K 31/4184; A61K 9/0053; A61K 9/08; A61K 31/70; A61K 31/7042; A61K 31/7048; A61K 31/7056; A61K 47/545; A61P 9/12; A61P 13/12

(57) ABSTRACT

The present invention is directed to the use of one or more SGLT-2 inhibitors or pharmaceutically acceptable forms thereof in combination with telmisartan or pharmaceutically acceptable forms thereof, in particular for the prophylaxis and/or treatment of one or more renal diseases and/or hypertension in a non-human mammal/non-human mammal patient, such as a dog or a cat.

### FIELD OF THE INVENTION

The invention relates to the field of medicine, in particular to the field of veterinary medicine. The invention relates to the combination treatment and/or prevention of renal diseases and/or hypertension in a non-human mammal, in particular a dog or a cat, comprising one or more SGLT-2 inhibitors or a pharmaceutically acceptable form thereof and telmisartan or a pharmaceutically acceptable form thereof.

BACKGROUND INFORMATION

Chronic kidney disease (CKD) is a major cause of morbidity and mortality in companion animals, especially in carnivores as the cat and the dog, with a prevalence of 1.0-3.0% in cats and 0.5-1.0% in dogs, increasing substantially in old age. Nephron damage associated with CKD is usually irreversible and often progressive.

The international renal interest society (IRiS) categorized CKD in different stages based initially on fasting blood creatinine or fasting blood symmetric dimethylarginine (SDMA) concentration or (preferably) both assessed on at least two occasions in a hydrated, stable patient. The patient is then sub-staged based on proteinuria and blood pressure into the following stages:
1—Normal blood creatinine or normal or mild increase in blood SDMA. Some other renal abnormality present (abnormal renal palpation, or renal imaging findings, proteinuria of renal origin, abnormal renal biopsy results, increasing blood creatinine or SDMA concentrations in samples collected serially). Persistently elevated blood SDMA concentration (>14 μg/dl) may be used to diagnose early CKD.
2—Normal or mildly increased creatinine, mild renal azotaemia (lower end of the range lies within reference ranges for creatinine for many laboratories, but the insensitivity of creatinine concentration as a screening test means that patients with creatinine values close to the upper reference limit often have excretory failure). Mildly increased SDMA. Clinical signs usually mild or absent.
3—Moderate renal azotemia. Many extrarenal signs may be present, but their extent and severity may vary. If signs are absent, the case could be considered as early Stage 3, while presence of many or marked systemic signs might justify classification as late Stage 3.
4—Increasing risk of systemic clinical signs and uremic crises.

Therapeutic standard of care usually consists of ACE inhibitors (e.g., enalapril), calcium channel blockers (e.g., diltiazem and amlodipine), angiotensin receptor blocker (e.g., telmisartan), targeting renal haemodynamics. In addition, clinical signs are treated as needed (e.g., vomiting, nausea, bodyweight loss, inappetence) and renal diets are available for the management of the disease.

State of the art interventions are based on specific action on unique pathways for symptomatic treatment of secondary conditions induced by renal conditions. In humans, beneficial renal effects under the use of SGLT-2 inhibitors were observed (DAPA-CKD ClinicalTrials.gov number, NCT03036150). However, the direct effect on kidney of carnivores like felines and canines remains unknown. Noteworthy, there are anatomical, physiological and pathophysiological differences between the species. First evidence for such differences becomes apparent by comparing the prevalence of CKD in cats compared to dogs (1.0-3.0% in cats and 0.5-1.0% in dogs) and humans (between 11 to 13% with the majority stage 3). Also, in contrast to people and dogs, primary glomerulopathies with marked proteinuria are remarkably rare findings in cats and the majority of geriatric cats with CKD do not have histologic evidence of primary glomerular disease. Typical histologic features include interstitial inflammation, tubular atrophy, and fibrosis with secondary glomerulosclerosis. In addition, in people, cardiovascular disease (CVD) is the primary cause of morbidity and mortality where CKD is regarded as an accelerator of CVD risk and an independent risk factor for CVD events. There is a graded inverse relationship between CVD risk and glomerular filtration rate (GFR) that is independent of age, sex and other risk factors. This is not the case in non-human mammals, e.g., carnivores, such as dogs and cats, and the pathogenesis of cardiovascular diseases (cardiomyopathies, valve diseases) differs significantly compared to the pathogenesis observed in people (coronary disease).

Systemic hypertension (SHT), which is synonymous with sustained increases in blood pressure (BP), is generally categorized into three types. It can be artificial/transient (stress-induced, situational), secondary to other disease processes that can increase blood pressure (secondary hypertension to e.g., CKD) or occur in the absence of other potentially causative disease (idiopathic hypertension).

Hypertension is a common comorbidity in human patients with diabetes type II (up to 86% of this patient cohort present hypertension) and is a major risk factor for the development of cardiac disease, chronic kidney disease, retinal degeneration and subsequent blindness, and stroke. The effects of SGLT-2 inhibition on BP is already recognized in these kinds of patients, however the exact mechanism of action remains elusive.

For example, the clinical trial EMPA-REG BP (N=825) reported that mean 24-hour systolic BP was significantly reduced from baseline to week 12 in both the empagliflozin 10 mg and 25 mg groups compared with the placebo group (placebo-subtracted mean difference up to −4.16 mmHg) in human patients suffering from diabetes mellitus type II. Similar results have been described for other SGLT-2 inhibitors.

Hypertension is a disease of modern civilization and heavily depends on environmental and particularly dietary factors of modern society. Specifically, the renin-angiotensin-aldosterone system (RAAS) has been adapted for sodium retention and has an important role in the pathogenesis of hypertension in human contemporary societies with high dietary salt intake (western diets). Besides, in humans there occurs smoking, lack of physical activity, alcohol abuse, stress, and sleep apnoea. The case is different with non-human mammals, especially carnivores, where hypertension is mostly associated with age and frequently diagnosed in association with other diseases such as chronic kidney disease and hyperthyroidism.

In non-human mammals such as the cat and the dog, the prevalence of hypertension depends on the presence of secondary disease linked to the condition. CKD, diabetes, hyperthyroidism, or obesity for example, are common causes of high blood pressure with a prevalence ranging from 46% in CKD patients to 15% in diabetic patients. Blood pressure increase is also linked to therapeutic treatments as glucocorticoids, mineralocorticoids, erythropoiesis-stimulating agents, ephedrine, and chronic high-dose sodium chloride. Idiopathic hypertension is chronic kidney disease more common than previously recognized, accounting for approximately 13%-20% of cases in cats and 24% in dogs (from a cohort of 42 animals presenting ocular hypertension).

Clinical signs of hypertension can include sudden blindness, bleeding inside the globe of the eye, and persistently dilated pupils, detached retinas, nervous system signs like depression, head tilt, seizures, disorientation, ataxia, weakness or partial paralysis and nystagmus. Additionally, increased fluid uptake and urinating with the progression of chronic kidney disease, hematuria, epistaxis and heart diseases have been reported.

The diagnosis of hypertension is based on direct blood pressure measurement, with direct arterial catheterization being the gold standard, but for practical reasons indirect BP measuring devices including oscillometry, high-definition oscillometry and doppler sphygmomanometry are preferred.

Typically, treatment starts at the hypertensive stage and the therapeutic goal is to bring blood pressure down to normotensive or pre-hypertensive levels, with the intention to reduce the risk of target organ damage (TOD). In the presence of TOD (e.g., heart and kidney disease) treatment should start immediately, after the initial measurement session. Otherwise, the diagnosis of hypertension should be based on at least two measurement sessions performed on different days.

The ACVIM panel recommends salt dietary restriction in addition to anti-hypertensive agents such as alpha and beta-adrenergic blockers, aldosterone receptor blockers, angiotensin converting enzyme inhibitors (ACEI), angiotensin receptor blockers, and calcium channel blockers depending on the primary cause of hypertension. Additional medications may be required depending on the response to initial therapy.

ACEIs are widely used as first-line treatment for SHT in dogs due to the role of the renin-angiotensin-aldosterone system (RAAS) in its development, but they provide an incomplete block of angiotensin II production that can result in poor control of SHT. This phenomenon, called “aldosterone breakthrough”, is due to the release of angiotensin II by other sites compared with those regulated by the ACE and independent of the dose of ACEI administered.

In humans, a reduction of blood pressure was observed using SGLT2-inhibitors in different clinical trials. However, the direct effect on blood pressure in non-human mammals like felines and canines remains unknown as there are anatomical, physiological, and pathophysiological differences between humans and non-human carnivores.

However, a beneficial effect of SGLT-2 inhibitors in carnivores, such as cats and dogs could be shown for the prevention and treatment of hypertension and also referring to renal diseases.

Angiotensin II plays an important part in pathophysiology, particularly as the most potent agent for increasing blood pressure in humans. It is known that in addition to its effect of raising blood pressure Angiotensin II also has growth-promoting effects which contribute to left ventricular hypertrophy, vascular thickening, atherosclerosis, renal failure and stroke. In small animals, inhibition of the effects of Angiotensin II, via either ACE inhibitors have been shown to exhibit renoprotective effects through their simultaneous capacity to decrease blood pressure and control proteinuria. Blockage of angiotensin II receptor 1 is a treatment concept which differs from blockage of angiotensin converting enzyme as known from the ACE-inhibitors. Receptor blockage is more specific and complete and further downstream in the physiologic cascade of the RAAS system. WO 2013/163675 discloses a method for the prophylaxis or treatment of a systemic disease in cats, wherein the method comprising administration of a therapeutically effective amount of angiotensin II receptor 1 (AT-1) antagonist (sartan) to that cat in need of such a treatment.

The angiotensin II receptor antagonist telmisartan (4′-[2-n-propyl-4-methyl-6-(1-methylbenzimidazol-2-yl)-benzimidazol-1-ylmethyl]-biphenyl-2-carboxylic acid) is a known pharmaceutically active compound to treat systemic diseases, such as chronic kidney disease, which was developed for the treatment of hypertension and other medical indications as disclosed in EP 0 502 314 and having the formula:

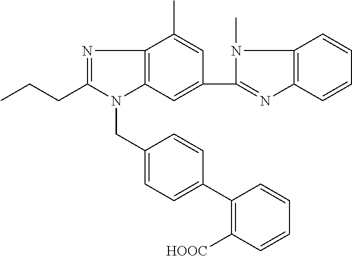 [SMILES: CCCc1nc2c(C)cc(-c3nc4ccccc4n3C)cc2n1Cc1ccc(-c2ccccc2C(=O)O)cc1]

Telmisartan is already sold on the market under the trade name Micardis® (Boehringer Ingelheim, Germany) for treatment/prophylaxis of humans. It exists in two polymorphic forms as disclosed in WO 00/043370, U.S. Pat. No. 6,358,986 and U.S. Pat. No. 6,410,742. Sodium salts of telmisartan and its solvate, hydrate, and hemihydrate are disclosed in WO 2003/037876. It is preferred to use telmisartan in its salt form, more preferably as a sodium salt.

Wheeler et al. (Diabetes Ther (2020) 11:2757-2774) reviews the actions of SGLT-2 inhibitors in chronic kidney disease progression in type 2 diabetes human patients.

Dekkers and Gansevoort (Nephrol Dial Transplant (2020) 35: i33-i42) describes the possible application of SGLT-2 inhibitors in non-diabetic kidney disease in humans.

Jepson RE (J Feline Med Surg. 2011; 13:25-34) describes classification and pathogenesis of feline systemic hypertension.

LeBlanc et al. (Journal of the American Veterinary Association. April 1, 2011, Vol. 238, No. 7, Pages: 915-921) analyses ocular lesions associated with systemic hypertension in dogs.

Acierno et al. (J Vet Intern Med. 2018: 32: 1803-1822.) describes guidelines for the identification, evaluation, and management of systemic hypertension in dogs and cats.

Ames et al. (Am J Vet Res 2015; 76:1041-50.) analyses the effects of high doses of enalapril and benazepril on the pharmacologically activated renin-angiotensin-aldosterone system in clinically normal dogs.

Atsuo T et al. (Naunyn-Schmiedeberg's Archives of Pharmacology 2018; 391 (4): 395-406) disclose the effects of the SGLT2 inhibitor ipragliflozin on various diabetic symptoms and progression of overt nephropathy in type 2 diabetic mice.

Shufei Z et al. (Biomedicine & Pharmacotherapy 2021, XP086927259) disclose the antifibrotic effects of low dose SGLT2 inhibition with empagliflozin in comparison to Ang II receptor blockade with telmisartan in 5/6 nephrectomised rats on high salt diet.

EP 3 508 222 discloses an agent or pharmaceutical composition for eliminating senescent cells, comprising an SGLT2-inhibitor.

US 2015/2792977 discloses SGLT2-inhibitors for use in the treatment and/or prevention of a metabolic disorder of an equine animal.

WO 2001/027128 discloses C-aryl glucoside SGLT2-inhibitors.

WO 2003/099836 discloses C-aryl glucoside SGLT2-inhibitors.

WO 2005/012326 discloses novel compounds having inhibitory activity against sodium dependent transporter.

WO 2007/140191 discloses glucose transport inhibitors and methods of use.

WO 2008/040774 A3 discloses a method of prophylaxis or treatment of systemic diseases in cats, wherein the method comprising administration of a therapeutically effective amount of angiotensin II receptor 1 (AT-1) antagonist (sartan) to a cat in need of such a treatment.

WO 2008/042688 discloses inhibitors of sodium glucose co-transporter 2 and methods of their use.

WO 2010/023594 discloses dioxa-bicyclo[3.2.1]octane-2,3,4-triol derivatives.

The International Patent application WO 2015/091313 A1 discloses the use of SGLT-2 inhibitors in or use in the treatment and/or prevention of a metabolic disorder in a feline animal.

The International Patent application WO 2015/110402 A1 discloses the use of SGLT-2 inhibitors in or use in the treatment and/or prevention of a metabolic disorder in a canine animal.

WO 2019/059557/US 2020/054656 discloses a pharmaceutical composition comprising an SGLT-2 inhibitor and a therapeutic agent for hypertension.

WO 2021/092341/US 2023/000816 discloses sodium-glucose linked transporter inhibitors for the management of chronic kidney disease, hypertension and heart failure in companion animals.

The International Patent application WO 2021/105152 A1 discloses the use of SGLT-2 inhibitors in the drying-off of non-human mammals.

WO 2022/036506 discloses a fixed dose combination of compositions of SGL T-2 inhibitors and angiotensin receptor blockers to prevent the worsening of renal failure for patients with chronic kidney disease.

WO 2023/006745 discloses the use of SGLT-2 inhibitors for the prevention and/or treatment of hypertension in non-human mammals.

WO 2023/006747 discloses the use of SGLT-2 inhibitors for the prevention and/or treatment of renal diseases in non-human mammals.

Notwithstanding the disclosures of the above documents, there is a medical need for the combination treatment and/or prevention of renal diseases and hypertension in a non-human mammal (patient), in particular a dog (patient) or a cat (patient).

### SUMMARY OF THE INVENTION

The present invention concerns one or more SGLT-2 inhibitors or pharmaceutically acceptable forms thereof in combination with an angiotensin II receptor 1 (AT-1) antagonist or a pharmaceutically acceptable form thereof for use as a medicament.

A comprehensive list of angiotensin II receptor antagonists can be found on pages 2 to 22 of WO 92/10182 and pages 7 to 18 of WO 95/26188, which all are incorporated herein by reference. Angiotensin II receptor antagonists are described inter alia in EP A 253310, EP-A-323841, EP-A-324377, EP-A-420237, EP-A-443983, EP A 459136, EP-A-475206, EP-A-502314, EP-A-504888, EP-A-514198, WO 91/14679, WO 93/20816, WO 02/092081, U.S. Pat. No. 4,355,040, U.S. Pat. No. 4,880,804 and U.S. Pat. No. 6,028,091. Forms which are frequently mentioned are sartans, such as candesartan, eprosartan, irbesartan, losartan, olmesartan, tasosartan, telmisartan or valsartan. Those which are particularly preferred according to the present invention are irbesartan, losartan und telmisartan. All of these sartans, or pharmaceutical salts or polymorphs thereof are well known to a person skilled in the art, and its use is within the meaning of the present invention.

Thus, the present invention relates to one or more SGLT-2 inhibitors or pharmaceutically acceptable forms thereof in combination with an angiotensin II receptor 1 (AT-1) antagonist or a pharmaceutically acceptable form thereof for use as a medicament, wherein the angiotensin II receptor 1 (AT-1) antagonist (sartan) is selected from the group consisting of: candesartan, eprosartan, irbesartan, losartan, olmesartan, tasosartan, telmisartan or valsartan, preferably of irbesartan, losartan or telmisartan.

More specifically, the present invention concerns one or more SGLT-2 inhibitors or pharmaceutically acceptable forms thereof in combination with telmisartan or pharmaceutically acceptable forms thereof for use as a medicament.

In one aspect, the present invention also concerns the one or more SGLT-2 inhibitors or pharmaceutically acceptable forms thereof in combination with an angiotensin II receptor 1 (AT-1) antagonist or a pharmaceutically acceptable form thereof for use as herein disclosed and/or claimed in a method of prevention and/or treatment of one or more renal diseases and/or hypertension in a non-human mammal/non-human mammal patient, in particular a canine/canine patient or a feline/feline patient.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with an angiotensin II receptor 1 (AT-1) antagonist or pharmaceutically acceptable forms thereof to such non-human mammal/non-human mammal patient as well as the corresponding use of one or more SGLT-2 inhibitors or pharmaceutically acceptable forms thereof in combination with telmisartan for the preparation of a medicament for the prevention and/or treatment of one or more renal diseases and/or hypertension in a non-human mammal/non-human mammal patient are also intended to be comprised by the present invention.

It is preferred, when the angiotensin II receptor 1 (AT-1) antagonist (sartan) is selected from the group consisting of: candesartan, eprosartan, irbesartan, losartan, olmesartan, tasosartan, telmisartan or valsartan, preferably of irbesartan, losartan or telmisartan or pharmaceutically acceptable forms thereof.

More specifically, the present invention also concerns the one or more SGLT-2 inhibitors or pharmaceutically acceptable forms thereof in combination with telmisartan or pharmaceutically acceptable forms thereof for use as herein disclosed and/or claimed in a method of prevention and/or treatment of one or more renal diseases and/or hypertension in a non-human mammal/non-human mammal patient, in particular a canine/canine patient or a feline/feline patient.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In another aspect, the present invention also concerns the one or more SGLT-2 inhibitors or pharmaceutically acceptable forms thereof in combination with telmisartan or pharmaceutically acceptable forms thereof for use as herein disclosed and/or claimed in a method of treatment of one or more renal diseases and/or hypertension in a non-human mammal/non-human mammal patient, in particular a canine/canine patient or a feline/feline patient.

A corresponding method of treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more renal diseases are selected from the group consisting of: renal dysplasia, glomerulopathy, polycystic kidney disease, amyloidosis, tubulo-nephritis/tubulointerstitial nephritis (TIN), acute kidney disease, chronic kidney disease, proteinuria.

A corresponding method of preventing and/or treating the above exemplified one or more renal diseases in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of the above exemplified one or more renal diseases in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more renal diseases are selected from the group consisting of: acute kidney disease, chronic kidney disease; and wherein the non-human mammal/non-human mammal patient is a canine/canine patient; preferably a canine patient in need of such prevention and/or treatment; more preferably a dog in need of such prevention and/or treatment.

A corresponding method of preventing and/or treating the above exemplified one or more renal diseases in a canine/canine patient, comprising administering one or more SGLT-2 inhibitors or pharmaceutically acceptable forms thereof in combination with telmisartan or pharmaceutically acceptable forms thereof to such canine/canine patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of the above exemplified one or more renal diseases in a canine/canine patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more renal diseases are selected from the group consisting of: acute kidney disease, chronic kidney disease; and wherein the non-human mammal/non-human mammal patient is a feline/feline patient; preferably a feline patient in need of such prevention and/or treatment; more preferably a cat in need of such prevention and/or treatment.

A corresponding method of preventing and/or treating the above exemplified one or more renal diseases in a feline/feline patient, comprising administering one or more SGLT-2 inhibitors or pharmaceutically acceptable forms thereof in combination with telmisartan or pharmaceutically acceptable forms thereof to such feline/feline patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of the above exemplified one or more renal diseases in a feline/feline patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the hypertension is selected from the group consisting of: systemic hypertension, glomerular hypertension, situational hypertension, secondary hypertension, and idiopathic hypertension.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of the above exemplified hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the hypertension is idiopathic hypertension.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the non-human mammal/non-human mammal patient, in particular a canine/canine patient or a feline/feline patient, more preferably a cat or a dog suffers from pre-hypertensive systolic blood pressure (SBP) with low target organ damage (TOD) risk, hypertensive SBP with moderate TOD risk or severely hypertensive SBP with high TOD risk.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns the one or more SGLT-2 inhibitors or pharmaceutically acceptable forms thereof as herein disclosed and/or claimed for use as a diuretic, optionally in combination with telmisartan or pharmaceutically acceptable forms thereof, in particular in the prevention and/or treatment of renal failure with oliguria or anuria and/or hypertension in a non-human mammal.

In one preferred embodiment, velagliflozin or pharmaceutically acceptable forms thereof is used as a diuretic, optionally in combination with telmisartan or pharmaceutically acceptable forms thereof, in particular in the prevention and/or treatment of renal failure with oliguria or anuria and/or hypertension. In another preferred embodiment, bexagliflozin or pharmaceutically acceptable forms thereof is used as a diuretic, optionally in combination with telmisartan or pharmaceutically acceptable forms thereof, in particular in the prevention and/or treatment of renal failure with oliguria or anuria and/or hypertension.

A corresponding method of using the one or more SGLT-2 inhibitors or pharmaceutically acceptable forms thereof as herein disclosed and/or claimed as a diuretic in a non-human mammal/non-human mammal patient, in particular in the prevention and/or treatment of renal failure with oliguria or anuria and/or hypertension in a non-human mammal/non-human mammal patient, in particular a canine/canine patient or a feline/feline patient, comprising administering one or more SGLT-2 inhibitors or pharmaceutically acceptable forms thereof, optionally in combination with telmisartan or pharmaceutically acceptable forms thereof, to such non-human mammal/non-human mammal patient as well as the corresponding use of one or more SGLT-2 inhibitors or pharmaceutically acceptable forms thereof, optionally in combination with telmisartan or pharmaceutically acceptable forms thereof, as a diuretic in a non-human mammal/non-human mammal patient, in particular for the preparation of a medicament for in the prevention and/or treatment of renal failure with oliguria or anuria and/or hypertension in a non-human mammal/non-human mammal patient are also intended to be comprised by the present invention.

In yet another aspect, the present invention also concerns the one or more SGLT-2 inhibitors or pharmaceutically acceptable forms thereof as herein disclosed and/or claimed in combination with one or more HIF prolyl-hydroxy lase inhibitors, selected from daprodustat, desidustat, enarodustat, molidustat, roxadustat and vadadustat for the prevention and/or treatment of renal diseases or related clinical signs, optionally in combination with telmisartan or pharmaceutically acceptable forms thereof, in particular in the prevention and/or treatment of renal diseases and/or hypertension in a non-human mammal, preferably a feline/feline patient. In a preferred embodiment, velagliflozin or pharmaceutically acceptable forms thereof is used in combination with one or more HIF prolyl-hydroxylase inhibitors optionally in combination with telmisartan or pharmaceutically acceptable forms thereof, in particular in the prevention and/or treatment of renal diseases and/or hypertension in a non-human mammal, preferably a feline/feline patient.

In another embodiment, velagliflozin or pharmaceutically acceptable forms thereof is used in combination with molidustat, optionally in combination with telmisartan or pharmaceutically acceptable forms thereof, in particular in the prevention and/or treatment of renal diseases and/or hypertension in a non-human mammal, preferably a feline/feline patient.

A corresponding method of using the one or more SGLT-2 inhibitors or pharmaceutically acceptable forms thereof as herein disclosed and/or claimed in combination with one or more HIF prolyl-hydroxylase inhibitors, selected from daprodustat, desidustat, enarodustat, molidustat, roxadustat and vadadustat for the prevention and/or treatment of renal diseases or related clinical signs, optionally in combination with telmisartan or pharmaceutically acceptable forms thereof, in particular in the prevention and/or treatment of renal diseases and/or hypertension in a non-human mammal, preferably a feline/feline patient, comprising administering one or more SGLT-2 inhibitors or pharmaceutically acceptable forms thereof in combination with one or more HIF prolyl-hydroxy lase inhibitors, selected from daprodustat, desidustat, enarodustat, molidustat, roxadustat and vadadustat, optionally in combination with telmisartan or pharmaceutically acceptable forms thereof, to such non-human mammal/non-human mammal patient as well as the corresponding use of one or more SGLT-2 inhibitors or pharmaceutically acceptable forms thereof in combination with one or more HIF prolyl-hydroxy lase inhibitors, selected from daprodustat, desidustat, enarodustat, molidustat, roxadustat and vadadustat, optionally in combination with telmisartan or pharmaceutically acceptable forms thereof, as a diuretic in a non-human mammal/non-human mammal patient, in particular for the preparation of a medicament for the prevention and/or treatment of renal diseases and/or hypertension in a non-human mammal/non-human mammal patient are also intended to be comprised by the present invention.

In yet another aspect, the present invention also concerns the following preferred embodiments:

One or more SGLT-2 inhibitors or pharmaceutically acceptable forms thereof in combination with telmisartan or pharmaceutically acceptable forms thereof for use in a method of prevention and/or treatment of CKD and/or hypertension in a canine/canine patient.

One or more SGLT-2 inhibitors or pharmaceutically acceptable forms thereof in combination with telmisartan or pharmaceutically acceptable forms thereof for use in a method of prevention and/or treatment of CKD and/or hypertension in a feline/feline patient.

One or more SGLT-2 inhibitors or pharmaceutically acceptable forms thereof in combination with telmisartan or pharmaceutically acceptable forms thereof for use in a method of treatment of CKD and/or hypertension in a canine/canine patient.

One or more SGLT-2 inhibitors or pharmaceutically acceptable forms thereof in combination with telmisartan or pharmaceutically acceptable forms thereof for use in a method of treatment of CKD and/or hypertension in a feline/feline patient.

In yet another aspect, the present invention also concerns the one or more SGLT-2 inhibitors or pharmaceutically acceptable forms thereof in combination with telmisartan or pharmaceutically acceptable forms thereof for the uses as herein disclosed and/or claimed, wherein the one or more SGLT-2 inhibitors are selected from the group consisting of:
(1) a glucopyranosyl-substituted benzene derivative of the formula (1)

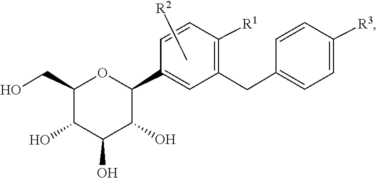

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxylethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R3 is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
(2) Velagliflozin, represented by formula (2):

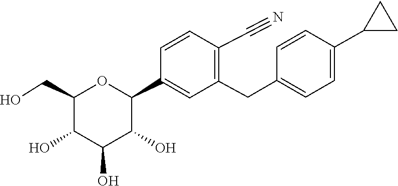 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

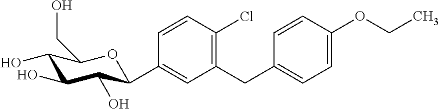

(4) Canagliflozin, represented by formula (4):

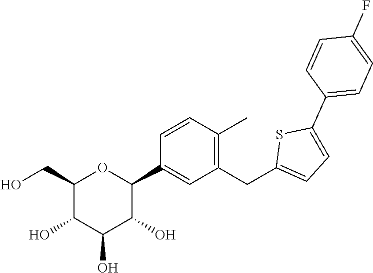 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

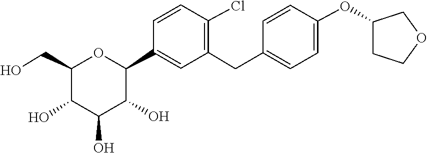 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

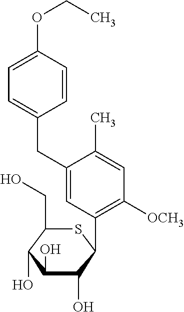

(7) Tofogliflozin, represented by formula (7):

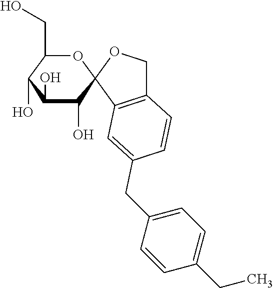

(8) Ipragliflozin, represented by formula (8):

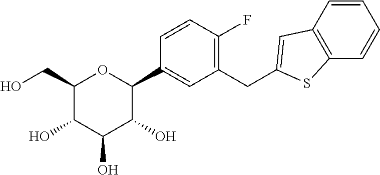 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

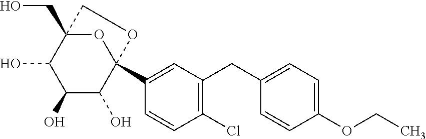

(10) Atigliflozin, represented by formula (10):

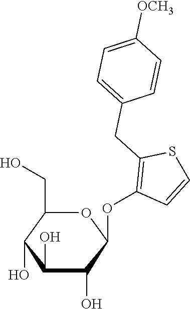

(11) Remogliflozin, represented by formula (11):

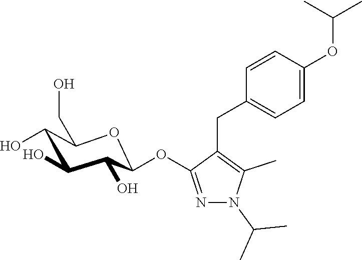

(11A) Remogliflozin etabonate, represented by formula (11A):

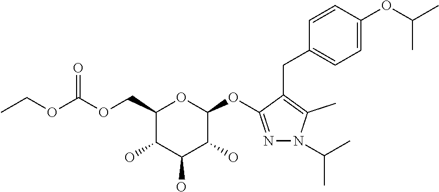 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

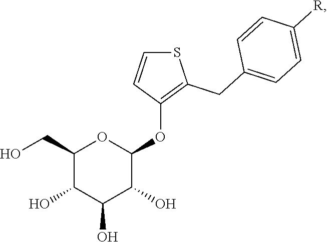

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

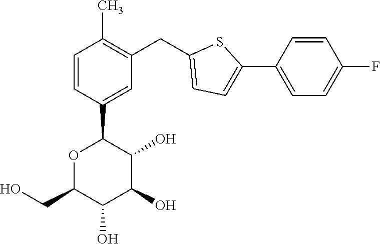 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

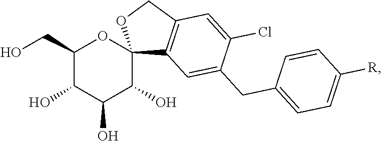

wherein R denotes methoxy, trifluoromethoxy, ethoxy, ethyl, isopropyl or tert. butyl;
(15) a pyrazole-O-glucoside derivative of the formula (15)

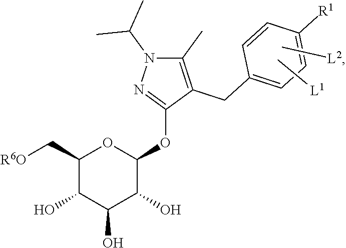

wherein
R¹ denotes C₁₋₃-alkoxy,
L¹, L² independently of each other denote H or F,
R⁶ denotes H, (C₁₋₃-alkyl) carbonyl, (C₁₋₆-alkyl) oxycarbonyl, phenyloxycarbonyl, benzyloxycarbonyl or benzylcarbonyl;
(16) Sotagliflozin, represented by formula (16):

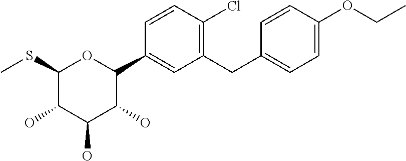

(17) Sergliflozin, represented by formula (17):

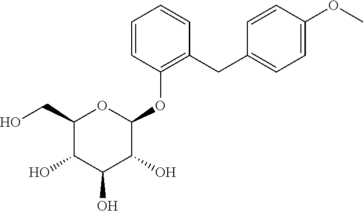 [SMILES: COc1ccc(Cc2ccccc2O[C@@H]2O[C@H](CO)[C@@H](O)[C@H](O)[C@H]2O)cc1]

(18) a compound represented by formula (18):

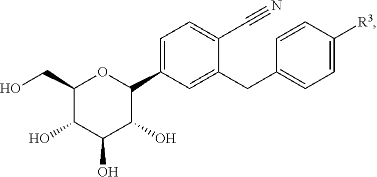

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
(19) Bexagliflozin, represented by formula (19):

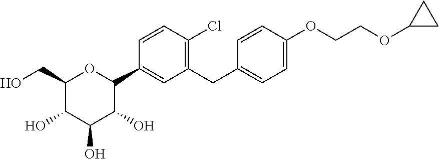

(20) Janagliflozin, represented by formula (20):

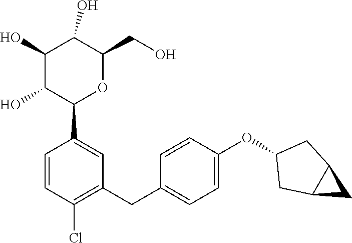 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

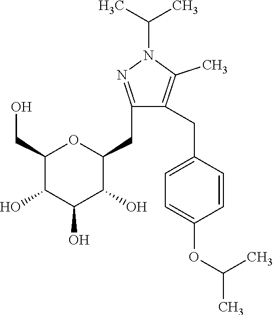 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by formula (23):

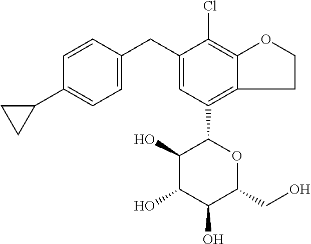 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

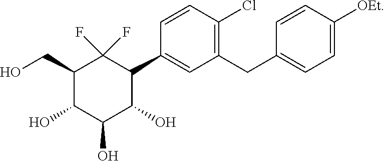

A corresponding method of preventing and/or treating one or more renal diseases and/or hypertension in a non-human mammal/non-human mammal patient, in particular a canine/canine patient or a feline/feline patient, comprising administering the above exemplified one or more SGLT-2 inhibitors or pharmaceutically acceptable forms thereof in combination with telmisartan or pharmaceutically acceptable forms thereof to such non-human mammal/non-human mammal patient as well as the corresponding use of the above exemplified one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the pharmaceutically acceptable form thereof is a crystalline complex between the one or more SGLT-2 inhibitors and one or more amino acids, preferably proline, more preferably L-proline; and most preferably is co-crystal of the one or more SGLT2 inhibitors, L-proline and crystalline water.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more SGLT-2 inhibitors is velagliflozin, which preferably is to be administered as single SGLT-2 inhibitor in combination with telmisartan or pharmaceutically acceptable forms thereof; or wherein the one or more SGLT-2 inhibitors is bexagliflozin, which preferably is to be administered as single SGLT-2 inhibitor in combination with telmisartan or pharmaceutically acceptable forms thereof.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

Accordingly, the present invention also concerns the following further preferred embodiments:

Velagliflozin or pharmaceutically acceptable forms thereof as single SGLT-2 inhibitor in combination with telmisartan or pharmaceutically acceptable forms thereof for use in a method of prevention and/or treatment of CKD and/or hypertension in a canine/canine patient.

Velagliflozin or pharmaceutically acceptable forms thereof as single SGLT-2 inhibitor in combination with telmisartan or pharmaceutically acceptable forms thereof for use in a method of prevention and/or treatment of CKD and/or hypertension in a feline/feline patient.

Velagliflozin or pharmaceutically acceptable forms thereof as single SGLT-2 inhibitor in combination with telmisartan or pharmaceutically acceptable forms thereof for use in a method of treatment of CKD and/or hypertension in a canine/canine patient.

Velagliflozin or pharmaceutically acceptable forms thereof as single SGLT-2 inhibitor in combination with telmisartan or pharmaceutically acceptable forms thereof for use in a method of treatment of CKD and/or hypertension in a feline/feline patient.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, in combination with one or more ACE inhibitors, which is preferably selected from benazepril, ramipril and enalapril. In another embodiment, velagliflozin or pharmaceutically acceptable forms thereof is combined with telmisartan or pharmaceutically acceptable forms thereof and benazepril. In another embodiment, velagliflozin or pharmaceutically acceptable forms thereof is combined with telmisartan or pharmaceutically acceptable forms thereof and ramipril. In another embodiment, velagliflozin or pharmaceutically acceptable forms thereof is combined with telmisartan or pharmaceutically acceptable forms thereof and enalapril. In another embodiment, bexagliflozin or pharmaceutically acceptable forms thereof is combined with telmisartan or pharmaceutically acceptable forms thereof and benazepril. In another embodiment, bexagliflozin or pharmaceutically acceptable forms thereof is combined with telmisartan or pharmaceutically acceptable forms thereof and ramipril. In another embodiment, bexagliflozin or pharmaceutically acceptable forms thereof is combined with telmisartan or pharmaceutically acceptable forms thereof and enalapril.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof and one or more ACE inhibitors to such non-human mammal/non-human mammal patient as well as the corresponding use of one or more SGLT-2 inhibitors or pharmaceutically acceptable forms thereof in combination with telmisartan or pharmaceutically acceptable forms and one or more ACE inhibitors for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more SGLT-2 inhibitors are administered orally, parenterally, intravenously, subcutaneously or intramuscularly, preferably orally.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more SGLT-2 inhibitors are to be administered at a dose of 0.01 mg/kg bodyweight to 10 mg/kg bodyweight per day, preferably at a dose of 0.01 mg/kg bodyweight to 5 mg/kg bodyweight per day, more preferably at a dose of 0.01 mg/kg bodyweight to 4 mg/kg bodyweight per day, even more preferably at a dose of 0.01 mg/kg bodyweight to 3 mg/kg bodyweight per day, even more preferably at a dose of 0.01 mg/kg bodyweight to 2 mg/kg bodyweight per day, even more preferably at a dose of 0.01 mg/kg bodyweight to 1 mg/kg bodyweight per day, even more preferably at a dose of 0.01 mg/kg bodyweight to 0.5 mg/kg bodyweight per day, most preferably at a dose of 0.01 mg/kg bodyweight to 0.3 mg/kg bodyweight per day. Alternatively, the one or more SGLT-2 inhibitors are to be administered at a dose of 0.1 mg/kg bodyweight to 10 mg/kg bodyweight per day, preferably at a dose of 0.1 mg/kg bodyweight to 5 mg/kg bodyweight per day, more preferably at a dose of 0.1 mg/kg bodyweight to 4 mg/kg bodyweight per day, even more preferably at a dose of 0.1 mg/kg bodyweight to 3 mg/kg bodyweight per day, even more preferably at a dose of 0.1 mg/kg bodyweight to 2 mg/kg bodyweight per day, even more preferably at a dose of 0.1 mg/kg bodyweight to 1 mg/kg bodyweight per day, even more preferably at a dose of 0.1 mg/kg bodyweight to 0.5 mg/kg bodyweight per day, most preferably at a dose of 0.1 mg/kg bodyweight to 0.3 mg/kg bodyweight per day.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein such one or more SGLT-2 inhibitors or pharmaceutically acceptable forms thereof is to be administered once per day or twice per day, preferably once per day.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein telmisartan is to be administered at a dose of about 0.01 to about 10 mg/kg of bodyweight per day. Preferably, said therapeutically effective amount is about 0.05 to about 8 mg/kg of bodyweight, even more preferably about 0.1 to about 5 mg/kg of bodyweight, even more preferably about 0.2 to about 4 mg/kg of bodyweight, even more preferably about 0.3 to about 3 mg/kg of bodyweight, even more preferably about 0.4 to about 2.5 mg/kg of bodyweight, even more preferably about 0.5 to about 2 mg/kg of bodyweight, most preferred about 0.75 to about 1.5 mg/kg of bodyweight per day. A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein telmisartan is to be administered once per day or twice per day, preferably once per day.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the one or more SGLT-2 inhibitors or pharmaceutically acceptable forms thereof are administered in combination with telmisartan or pharmaceutically acceptable forms thereof, preferably wherein the one or more SGLT-2 inhibitors or pharmaceutically acceptable forms thereof is to be administered before, after or concomitantly with administering telmisartan or pharmaceutically acceptable forms thereof.

A corresponding method of preventing and/or treating one or more renal diseases and/or hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases and/or hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the preventive and/or therapeutic effect is characterized by one or more of the following clinical and/or biochemical parameters:
improved renal efficiency, characterized by a reduction of proteinuria-and/or a reduction and/or stabilization of blood parameters such as serum SDMA, serum creatinine, FGF23, blood urea nitrogen (BUN) and/or hyperphosphatemia, and/or slowed reduction in glomerular filtration rate (GFR);
increase of the production of ketone bodies in the liver, characterized by increased plasma levels of 3-hydroxybutyric acid and/or the corresponding acylcarnitines i.e., hydroxybutyrylcarnitine and increased plasma levels of one or more of the branched-chain amino acids (e.g., valine, leucine and isoleucine);
delayed onset of hypertension and/or prevention of target organ damage and/or improved blood pressure;
improved hydration status;
delayed onset of renal failure, preferably at least by 1, 2, 3, 4, 5, 6, 7, 8, 9, 10, 11, 12 or more months, or delayed and/or stopped progression of the one or more renal diseases, in particular chronic kidney disease, and/or improvement of the classification stage of the one or more renal diseases, in particular CKD (e.g., from stage III to stage II);
promoting diuresis in order to reduce renal failure with oliguria or anuria and/or hypertension;
longer survival time of the non-human mammal patient, preferably at least by 1, 2, 3, 4, 5, 6, 7, 8, 9, 10, 11, 12 or more months and/or lower renal related mortality and/or morbidity;
improved clinical signs, such as reduced polydipsia, polyuria, vomiting, lethargy, weight loss, and/or signs associated with dehydration;
higher quality of life.

A corresponding method of preventing and/or treating one or more renal diseases in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of one or more renal diseases in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the systolic blood pressure (SBP) value measured for the non-human mammal/non-human mammal patient, in particular a canine/canine patient or a feline/feline patient, more preferably a cat or a dog, in need thereof is decreased after the period of the treatment by at least 5 mmHg, preferably by at least 10 mmHg, more preferably by at least 20 mmHg, in particular by 5 to 100 mmHg, more preferably 5 to 50 mmHg, most preferably by 10 to 50 mmHg, in relation to the baseline SBP value measured for the non-human mammal/non-human mammal patient, in particular a canine/canine patient or a feline/feline patient, more preferably a cat or a dog, prior to the period of treatment.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the method comprises measurement of the SBP and optionally identification of TOD followed by administration of a therapeutically effective amount of the one or more SGLT-2 inhibitors or pharmaceutically acceptable forms thereof to the non-human mammal/non-human mammal patient, in particular a canine/canine patient or a feline/feline patient, more preferably a cat or a dog, wherein the therapeutically effective amount of the one or more SGLT-2 inhibitors or pharmaceutically acceptable forms thereof in combination with telmisartan or pharmaceutically acceptable forms thereof is administered in a daily dosage amount that may be varied over a treatment period depending on subsequent measurements of the SBP.

A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns the one or more SGLT-2 inhibitors or pharmaceutically acceptable forms thereof in combination with telmisartan or pharmaceutically acceptable forms thereof for use as herein disclosed and/or claimed, wherein the hypertension is non-refractory to the treatment with ACE inhibitors in the non-human mammal/non-human mammal patient, in particular a canine/canine patient or a feline/feline patient, in particular the cat or dog to be treated, most preferably the dog to be treated. A corresponding method of preventing and/or treating hypertension in a non-human mammal/non-human mammal patient, in particular a canine/canine patient or a feline/feline patient, comprising administering one or more SGLT-2 inhibitors or pharmaceutically acceptable forms thereof in combination with telmisartan or pharmaceutically acceptable forms thereof to such non-human mammal/non-human mammal patient as well as the corresponding use of one or more SGLT-2 inhibitors or pharmaceutically acceptable forms thereof in combination with telmisartan or pharmaceutically acceptable forms thereof for the preparation of a medicament for the prevention and/or treatment of hypertension in a non-human mammal/non-human mammal patient are also intended to be comprised by the present invention.

In yet another aspect, the present invention also concerns a pharmaceutical composition comprising one or more SGLT2 inhibitors or pharmaceutically acceptable forms thereof in combination with telmisartan or pharmaceutically acceptable forms thereof, wherein preferably such pharmaceutical composition is a fixed-dose-combination (FDC) of the one or more SGLT-2 inhibitors or pharmaceutically acceptable forms thereof in combination with telmisartan or pharmaceutically acceptable forms thereof, wherein more preferably such FDC is a solid or liquid formulation.

The advantages according to the present invention are one or more of the following:
dose reduction of the individual active ingredients (independently from each other) and/or replacement of further concomitant treatments (e.g., ACEi, Ca²⁺-channel blocker) as compared to single respective treatment,
dose reduction of further concomitant treatments (e.g., ACEi) as compared to standard of care treatment,
improved renal efficacy, increased renal health via protection of e.g., proximal tubular cells,
reduction of intra-glomerular pressure, specifically higher reduction of intra-glomerular pressure leading to reduction of proteinuria,
protection of renal nephrons, prevention of glomerulonephritis and tubulonephritis, interstitial inflammation (fibrosis),
prevention of progression of disease, especially later hospitalization and intensive care, reduced target organ damage,
less side effects (e.g., hospitalization),
prolongation of survival time.

### DETAILED DESCRIPTION OF THE INVENTION

Before the embodiments of the present invention are described in further detail, it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In a preferred embodiment, the “non-human mammal” is selected from the group consisting of: bovine, canine, caprine, equine, feline, lagomorphs, ovine, porcine, rodent; more preferably is selected from the group consisting of: cattle, cow, dog, cat, goat, horse, pony, donkey, sheep, pig, rabbit, rat, mouse; even more preferably selected from the group consisting of: canine or feline; most preferably selected from the group consisting of: dog or cat.

In the course of the present invention, the term “canine animal” or “canine” refers to any member of the canidae family (i.e., a canid). It may thus belong either to the subfamily canidae or the subfamily caninae. The term canine animal encompasses the term dog, e.g., a domestic dog. The term domestic dog encompasses the terms Canis familiaris or Canis lupus familiaris. Most preferably, the canine animal or canine is a dog, in particular a domestic dog.

In the course of the present invention, the term “feline animal” or “feline” refers to any member of the Felidae family (i.e., a felid). It may thus belong either to the subfamily felinae or the subfamily pantherinae. The term feline animal encompasses the term cat, e.g., a domestic cat. The term domestic cat encompasses the terms Felis catus and Felis silvestris catus. Most preferably, the feline animal or feline is a cat, in particular a domestic cat.

In the course of the present invention, the term “renal dysplasia” refers to a rare renal malformation in which the kidney(s) are present, but their development is abnormal, leading to malformation of histologic architecture of the kidney.

In the course of the present invention, the term “glomerulopathy” refers to the occurrence of glomerulonephritis with or without an accompanying condition.

In the course of the present invention, the term “polycystic kidney disease” refers to an inherited disorder in which liquid-filled sacs develop in the tissue of the kidney.

In the course of the present invention, the term “amyloidosis” refers to a group of diseases in which abnormal proteins, known as amyloid fibrils, build up in (kidney) tissue.

In the course of the present invention, the term “acute kidney disease” refers to a sudden episode of kidney failure or kidney damage.

In the course of the present invention, the terms “tubulo-nephritis” and “tubulointerstitial nephritis (TIN)” are used interchangeably and refer to a frequent cause of acute kidney injury (AKI) that can lead to chronic kidney disease (CKD). TIN is associated with an immune-mediated infiltration of the kidney interstitium by inflammatory cells, which may progress to fibrosis.

In the course of the present invention, the term “chronic kidney disease (CKD)” refers to the pathophysiolog ical process, in which the remaining renal function is not enough to maintain the glomerular filtration rate at physiologic levels. It can also be defined as a complex clinical syndrome, that is based on abnormal structure and function of the kidneys and is characterized by weight loss, halitosis, poor hair quality, proteinuria, high blood pressure, azotaemia, polyuria, polydipsia, vomiting and anaemia. It is mostly a chronic condition, due to a progression in nephron damage, initiated by different factors, including diabetes, ischemic insults, toxic insults, some viral infections and cardiac conditions leading to high blood pressure.

As used herein, the term “hypertension” refers to an elevated pressure of the blood against the walls of arteries during the time the heart contracts and empties itself of blood as well as during the time the heart relaxes and fills with blood and in. The term embraces systemic hypertension and idiopathic hypertension.

The term “systemic hypertension” or abbreviated SHT is applied to sustained increases in systolic blood pressure (SBP>140 mmHg), and generally can be categorized into 1 of 3 types: (i) it may be caused by environmental or situational stressors, (ii) it may occur in association with other disease processes that increase BP (ie, secondary hypertension), or (iii) it may occur in the absence of other potentially causative disease processes (ie, idiopathic hypertension).

“SHT in cats and dogs is classified based on the risk of target organ damage (TOD) according to the ACVIM consensus statement (Ref. 3), as follows:
Normotensive (minimal TOD risk) systolic BP<140 mm Hg
Pre-hypertensive (low TOD risk) systolic BP 140-159 mmHg
Hypertensive (moderate TOD risk) systolic BP 160-179 mm Hg
Severely hypertensive (high TOD risk) systolic BP>180 mm Hg.

As used herein, the term “non-refractory to the treatment with ACE inhibitors (ACEI)” refers to non-human mammals, preferably carnivores, in particular cats or dogs suffering from hypertension, which can be treated with an ACEI, but with less efficacy than an SGLT-2 inhibitor. To the contrary the high values of sustained systolic arterial blood pressure (SBP) of non-human mammals that are refractory to ACEIs cannot be lowered with the aid of ACEIs.

In the non-refractory sub-population of non-human mammals the efficacy of treatment with an ACEI is 10%, 15%, 20%, 25%, 30%, 35%, 40%, 45%, 50%, more than 50%, more than 60%, or more than 70% less effective than an SGLT-2 inhibitor for lowering their SBP values.

In a preferred embodiment the one or more SGLT-2 inhibitor according to the invention relates to the treatment and/or prevention of the non-refractory subpopulation of dogs.

In the course of the present invention, the term “one or more SGLT-2 inhibitors or pharmaceutically acceptable forms thereof in combination with telmisartan” relates to the medical combination of the two or more active ingredients, i.e., the one or more SGLT-2 inhibitors or pharmaceutically acceptable forms thereof are administered to the non-human mammal (patient) before, after or concomitantly with administering telmisartan. In this context, the two or more active ingredients can be present in the same dosage form and as a result are concomitantly administered. Alternatively, the two or more active ingredients can be present in separate (identical or different) dosage forms for administration after one another at different time points (“before” or “after” aspects) or at the same time point (“concomitantly” aspect).

SGLT-2 inhibitors for use according to the invention include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Moreover, the one or more SGLT-2 inhibitors for use according to the invention may be selected from the group consisting of the following compounds or pharmaceutically acceptable forms thereof:
(1) a glucopyranosyl-substituted benzene derivative of the formula (1)

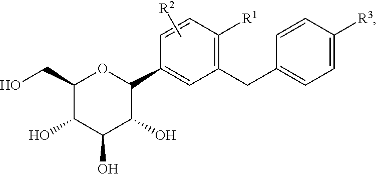

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxylethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
(2) Velagliflozin, represented by formula (2):

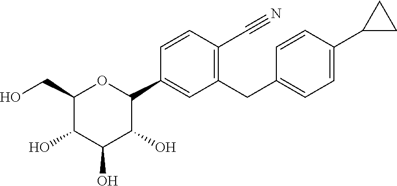

(3) Dapagliflozin, represented by formula (3):

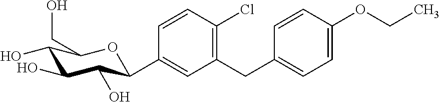

(4) Canagliflozin, represented by formula (4):

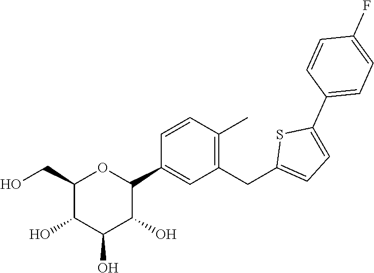

Empagliflozin, represented by formula (5):
(5) Empagliflozin, represented by formula (5):

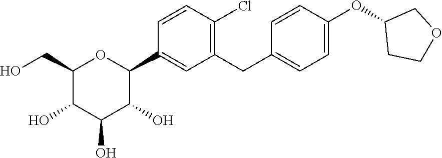 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

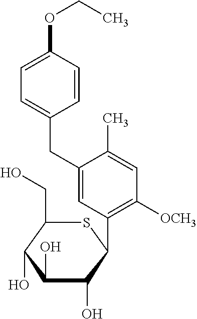

(7) Tofogliflozin, represented by formula (7):

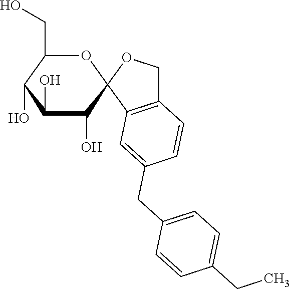

(8) Ipragliflozin, represented by formula (8):

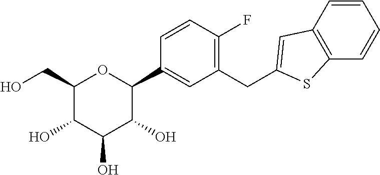 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

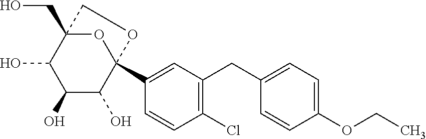

(10) Atigliflozin, represented by formula (10):

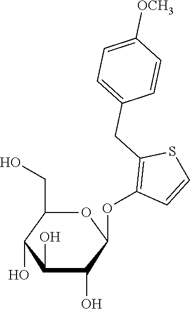

(11) Remogliflozin, represented by formula (11):

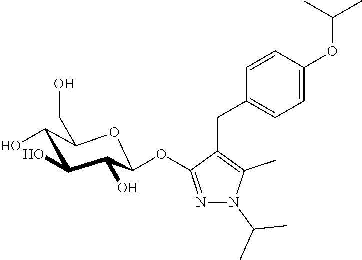

(11A) Remogliflozin etabonate, represented by formula (11A):

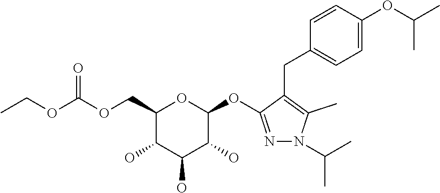 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

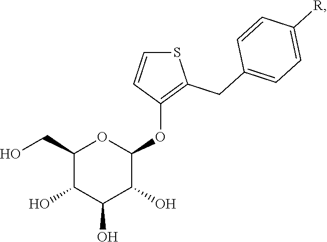

wherein R denotes methoxy or trifluoromethoxy;
(13) 1-(β-D)-glucopyranosyl)-4-methyl-3-[5-(4-fluorophenyl)-2-thienylmethyl]benzene, represented by formula (13);

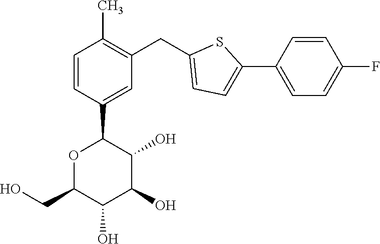 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

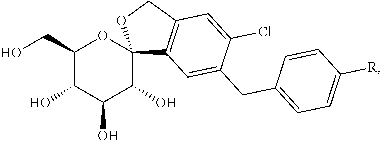

wherein R denotes methoxy, trifluoromethoxy, ethoxy, ethyl, isopropyl or tert. butyl;
(15) a pyrazole-O-glucoside derivative of the formula (15)

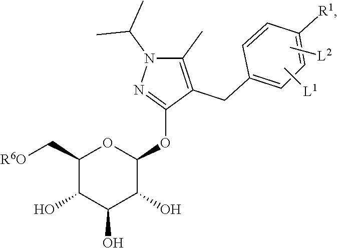

wherein
R¹ denotes C₁₋₃-alkoxy,
L¹, L² independently of each other denote H or F,
R⁶ denotes H, (C₁₋₃-alkyl) carbonyl, (C₁₋₆-alkyl) oxycarbonyl, phenyloxycarbonyl, benzyloxycarbonyl or benzylcarbonyl;
(16) Sotagliflozin, represented by formula (16):

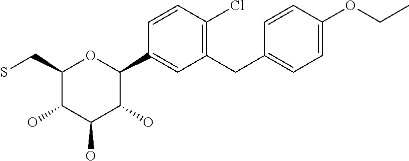 [SMILES: CCOc1ccc(Cc2cc([C@@H]3O[C@H](CS)[C@@H](O)[C@H](O)[C@H]3O)ccc2Cl)cc1]

(17) Sergliflozin, represented by formula (17):

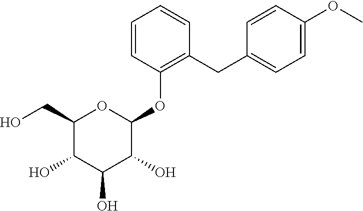 [SMILES: COc1ccc(Cc2ccccc2O[C@@H]2O[C@H](CO)[C@@H](O)[C@H](O)[C@H]2O)cc1]

(18) a compound represented by formula (18):

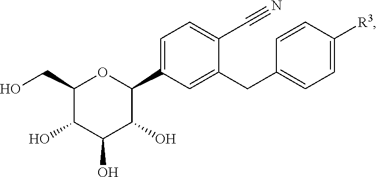

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
(19) Bexagliflozin, represented by formula (19):

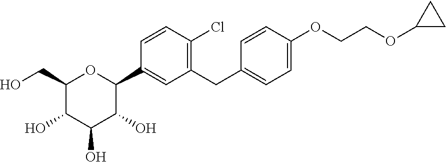 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

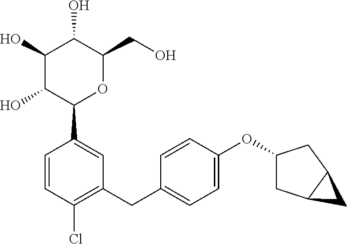 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

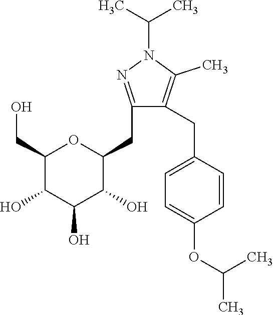 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by formula (23):

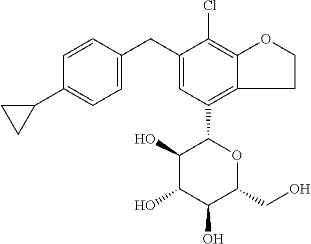 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

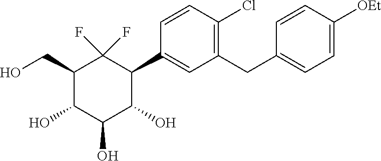 [SMILES: CCOc1ccc(Cc2cc([C@H]3[C@H](O)[C@@H](O)[C@H](O)[C@@H](CO)C3(F)F)ccc2Cl)cc1]

The term “velagliflozin” as employed herein refers to velagliflozin of the above structure as well as pharmaceutically acceptable forms thereof, including hydrates and solvates thereof, and crystalline forms thereof.

The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof. The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof.

The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e., sotagliflozin, and its manufacture are described for example in WO 2008/042688 or WO 2009/014970.

The term “bexagliflozin” as employed herein refers to bexagliflozin of the above structure as well as pharmaceutically acceptable forms thereof, including hydrates and solvates thereof, and crystalline forms thereof. The compound and methods of its synthesis are described in WO 2009/026537 for example.

The term “TFC-039” as employed herein refers to the above structure as well as pharmaceutically acceptable forms thereof, including hydrates and solvates thereof, and crystalline forms thereof. The compound and methods of its synthesis are described in WO 2012/160218 for example.

Preferred SGLT-2 inhibitors are glucopyranosyl-substituted benzene derivatives. Optionally, one or more hydroxyl groups of the glucopyranosyl group in such one or more SGLT-2 inhibitors may be acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl.

More preferred are glucopyranosyl-substituted benzonitrile derivatives of formula (1) as disclosed herein above. Yet more preferred are glucopyranosyl-substituted benzonitrile derivatives of formula (18):

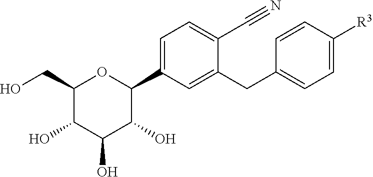

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxypropyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl.

Preferably, such SGLT-2 inhibitor is velagliflozin as shown in formula (2). Optionally, one or more hydroxyl groups of the β-D-glucopyranosyl group of velagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in a preferred embodiment, the at least one SGLT-2 inhibitor according to the present invention is a glucopyranosyl-substituted benzene derivative SGLT-2 inhibitor, preferably a SGLT-2 inhibitor of formula (1), more preferably of formula (18), or yet more preferably of formula (2), i.e., velagliflozin, in each case as defined herein above.

In another preferred embodiment, such SGLT-2 inhibitor is bexagliflozin as shown in formula (19). Optionally, one or more hydroxyl groups of the β-D-glucopyranosyl group of bexagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in another preferred embodiment, the at least one SGLT-2 inhibitor according to the present invention is a glucopyranosyl-substituted benzene derivative SGLT-2 inhibitor, preferably an SGLT-2 inhibitor of formula (19), i.e., bexagliflozin, in each case as defined herein above.

Herein, references to SGLT-2 inhibitors and/or their use according to the invention encompass pharmaceutically acceptable forms of the SGLT-2 inhibitors, unless otherwise stated.

According to the invention, any pharmaceutically acceptable form of the SGLT-2 inhibitor, e.g., of formula (1), preferably formula (18), more preferably formula (2), may be used. E.g., a crystalline form may be used. Prodrug forms are also encompassed by the present invention.

Prodrug forms may include, e.g., esters and/or hydrates. The term “prodrug” is also meant to include any covalently bonded carrier, which releases the active compound of the invention in vivo when the prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention.

Crystalline forms for use according to the invention include a complex of an SGLT-2 inhibitor with one or more amino acids (see e.g., WO 2014/016381)—so-called co-crystals. An amino acid for such use may be a natural amino acid. The amino acid may be a proteogenic amino acid (including L-hydroxyproline), or a non-proteogenic amino acid. The amino acid may be a D- or an L-amino acid. In some preferred embodiments, the amino acid is proline (L-proline and/or D-proline, preferably L-proline). E.g., a crystalline complex/co-crystal of velagliflozin with proline (e.g., L-proline) and crystalline water is preferred.

Thus, herein is disclosed a crystalline complex/co-crystal between one or more natural amino acids and an SGLT-2 inhibitor, e.g., a crystalline complex/co-crystal between one or more natural amino acids and a glucopyranosyl-substituted benzene derivative SGLT-2 inhibitor, preferably a SGLT-2 inhibitor of formula (1), more preferably of formula (18) or yet more preferably of formula (2) (velagliflozin).

A certain pharmaceutical activity is the basic prerequisite to be fulfilled by a pharmaceutically active agent before it is approved as a medicament on the market. However, there are a variety of additional requirements a pharmaceutically active agent has to comply with. These requirements are based on various parameters, which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions, which contain, in addition to the actual active substance, breakdown products thereof, for example. In such cases, the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g., by grinding. Since breakdown of the pharmaceutically active substance as a side effect of the grinding (or micronizing) has to be avoided as far as possible, in spite of the hard conditions required during the process, it is essential that the active substance should be highly stable throughout the grinding process. Only if the active substance is sufficiently stable during the grinding process it is possible to produce a homogeneous pharmaceutical formulation, which always contains the specified amount of active substance in a reproducible manner.

Another problem, which may arise in the grinding process for preparing the desired pharmaceutical formulation, is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament; the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance because of the increased weight caused by the uptake of water. Pharmaceutical compositions with a tendency to absorb moisture have to be protected from moisture during storage, e.g., by the addition of suitable drying agents or by storing the drug in an environment where it is protected from moisture. Preferably, therefore, a pharmaceutically active substance should be at best slightly hygroscopic.

Furthermore, the availability of a well-defined crystalline form allows the purification of the drug substance by recrystallization.

Apart from the requirements indicated above, it should be generally borne in mind that any change to the solid state of a pharmaceutical composition, which is capable of improving its physical and chemical stability, gives a significant advantage over less stable forms of the same medicament.

A crystalline complex/co-crystal between a natural amino acid and an SGLT-2 inhibitor (e.g., a glucopyranosyl-substituted benzene derivative or a SGLT-2 inhibitor of formula (1), or formula (18) or, particularly, of formula (2), i.e., velagliflozin) fulfills important requirements mentioned hereinbefore.

SGLT-2 inhibitors or pharmaceutically acceptable forms thereof for use according to the invention may be prepared as pharmaceutical compositions. They may be prepared as solid or as liquid formulations. In either case, they are preferably prepared for oral administration, preferably in liquid form for oral administration (see e.g., WO 2017/032799). The SGLT-2 inhibitors or pharmaceutically acceptable forms thereof may, however, also be prepared, e.g., for parenteral administration. Solid formulations include tablets, granular forms, and other solid forms such as suppositories. Among solid formulations, tablets and granular forms are preferred.

Telmisartan for use according to the invention may be prepared as pharmaceutical compositions. It may be prepared as solid or as liquid formulations. In either case, it is preferably prepared for oral administration, preferably in solid form (tablets) for oral administration (see e.g., WO 2004/028505 or WO 2004/096215).

Pharmaceutical compositions within the meaning of the present invention may comprise one or more SGLT-2 inhibitors or pharmaceutically acceptable forms thereof according to the present invention in combination with telmisartan or pharmaceutically acceptable forms thereof and one or more excipients. Any excipient that allows for, or supports, the intended medical effect may be used. Such excipients are available to the skilled person. Useful excipients are for example anti-adherents (used to reduce the adhesion between the powder (granules) and the punch faces and thus prevent sticking to tablet punches), binders (solution binders or dry binders that hold the ingredients together), coatings (to protect tablet ingredients from deterioration by moisture in the air and make large or unpleasant-tasting tablets easier to swallow), disintegrants (to allow the tablet to break upon dilution), fillers, diluents, flavours, colours, glidants (flow regulators—to promote powder flow by reducing interparticle friction and cohesion), lubricants (to prevent ingredients from clumping together and from sticking to the tablet punches or capsule filling machine), preservatives, sorbents, sweeteners etc.

Formulations according to the invention, e.g., solid formulations, may comprise carriers and/or disintegrants selected from the group of sugars and sugar alcohols, e.g., mannitol, lactose, starch, cellulose, microcrystalline cellulose and cellulose derivatives, e.g., methylcellulose, and the like.

Manufacturing procedures for formulations suitable for canines are known to the person skilled in the art, and for solid formulations comprise, e.g., direct compression, dry granulation and wet granulation. In the direct compression process, the active ingredient and all other excipients are placed together in a compression apparatus that is directly applied to press tablets out of this material. The resulting tablets can optionally be coated afterwards in order to protect them physically and/or chemically, e.g., by a material known from the state of the art.

A unit for administration, e.g. a single liquid dose or a unit of a solid formulation, e.g. a tablet, may comprise 0.1 mg to 10 mg, or e.g., 0.3 mg to 1 mg, 1 mg to 3 mg, 3 mg to 10 mg; or 5 to 2500 mg, or e.g., 5 to 2000 mg, 5 mg to 1500 mg, 10 mg to 1500 mg, 10 mg to 1000 mg, or 10-500 mg of an SGLT-2 inhibitor or pharmaceutically acceptable forms thereof as well as 0.03 mg to 10 mg telmisartan, or e.g., 1.25 mg, 2.5 mg, 5 mg or 10 mg telmisartan or pharmaceutically acceptable forms thereof for use according to the invention. As the skilled person would understand, the content of the SGLT-2 inhibitor or pharmaceutically acceptable forms thereof and telmisartan or pharmaceutically acceptable forms thereof in a solid formulation, or any formulation as disclosed herein for administration to a non-human mammal, may be increased or decreased as appropriate in proportion to the bodyweight of the non-human mammal to be treated.

In one embodiment, a pharmaceutical composition for use according to the invention is designed for oral or parenteral administration, preferably for oral administration. Especially the oral administration is ameliorated by excipients, which modify the smell and/or haptic properties of the pharmaceutical composition for the intended patient, e.g., as described.

When the SGLT-2 inhibitor or pharmaceutically acceptable forms thereof in combination with telmisartan or pharmaceutically acceptable forms thereof for use according to the invention is formulated for oral administration, it is preferred that excipients confer properties, e.g., palatability and/or chewability that render the formulation suitable for administration to a non-human mammal.

Also preferred are liquid formulations. Liquid formulations may be, e.g., solutions, syrups or suspensions. They may be administered directly to the non-human mammal or may be mixed with the food and/or drink (e.g., drinking water, or the like) of the non-human mammal. One advantage of a liquid formulation (similar to a formulation in granular form), is that such a dosage form allows precise dosing. For example, the SGLT-2 inhibitor or pharmaceutically acceptable forms thereof and telmisartan or pharmaceutically acceptable forms thereof may be dosed precisely in proportion to the body mass of a non-human mammal. Typical compositions of liquid formulations are known to the person skilled in the art.

According to the present invention two or more pharmaceutical active substances can be combined in one single dosage form, i.e., as combination drugs. The advantage of such a formulation is that the doses are fixed in this pharmaceutical formulation, i.e., available in certain fixed doses. In such a case the pharmaceutical formulation is called a “fixed-dose-combination” (FDC), which can be either a solid or a liquid formulation.

In another embodiment, a pharmaceutical composition for use according to the invention is a fixed-dose-combination (FDC) of the one or more SGLT-2 inhibitors or pharmaceutically acceptable forms thereof and telmisartan or pharmaceutically acceptable forms thereof, wherein preferably such FDC is a solid or a liquid formulation.

In a preferred embodiment, the FDC comprises velagliflozin or pharmaceutically acceptable forms thereof and telmisartan or pharmaceutically acceptable forms thereof as the only pharmaceutical active substances. In another preferred embodiment, the FDC comprises bexagliflozin or pharmaceutically acceptable forms thereof and telmisartan or pharmaceutically acceptable forms thereof as the only pharmaceutical active substances.

A practitioner skilled in the art can determine suitable doses for the uses of the present invention. Preferred units dosing units include mg/kg bodyweight, i.e., mg SGLT-2 inhibitor per body mass of the non-human mammal. An SGLT-2 inhibitor of the invention may, e.g., be administered in doses of 0.01-10 mg/kg bodyweight per day, e.g., 0.01-5 mg/kg bodyweight per day, e.g., 0.01-4 mg/kg bodyweight per day, e.g., 0.01-3 mg/kg bodyweight per day, e.g., 0.01-2 mg/kg bodyweight per day, e.g., 0.01-1.5 mg/kg bodyweight per day, e.g., 0.01-1 mg/kg bodyweight per day, e.g., 0.01-0.75 mg/kg bodyweight per day, e.g., 0.01-0.5 mg/kg bodyweight per day, e.g., 0.01-0.4 mg/kg bodyweight per day; or 0.1 to 3.0 mg/kg bodyweight per day, preferably from 0.2 to 2.0 mg/kg bodyweight per day, more preferably from 0.1 to 1 mg/kg bodyweight per day or from 0.5 to 1 mg/kg bodyweight per day. In another preferred embodiment, the dose is 0.01-1 mg/kg bodyweight per day, preferably 0.01-0.5 mg/kg bodyweight per day, more preferably 0.02-0.4 mg/kg bodyweight per day, e.g. 0.03-0.3 mg/kg bodyweight per day. Alternatively, an SGLT-2 inhibitor of the invention may, e.g., be administered in doses of 0.1 mg/kg bodyweight to 10 mg/kg bodyweight per day, preferably at a dose of 0.1 mg/kg bodyweight to 5 mg/kg bodyweight per day, more preferably at a dose of 0.1 mg/kg bodyweight to 4 mg/kg bodyweight per day, even more preferably at a dose of 0.1 mg/kg bodyweight to 3 mg/kg bodyweight per day, even more preferably at a dose of 0.1 mg/kg bodyweight to 2 mg/kg bodyweight per day, even more preferably at a dose of 0.1 mg/kg bodyweight to 1 mg/kg bodyweight per day, even more preferably at a dose of 0.1 mg/kg bodyweight to 0.5 mg/kg bodyweight per day, most preferably at a dose of 0.1 mg/kg bodyweight to 0.3 mg/kg bodyweight per day.

A practitioner skilled in the art is able to prepare an SGLT-2 inhibitor of the invention for administration according to a desired dose.

As regards telmisartan or pharmaceutically acceptable forms thereof, a practitioner skilled in the art can determine suitable doses for the uses of the present invention. Preferred units dosing units include mg/kg bodyweight, i.e., mg telmisartan per body mass of the non-human mammal. Telmisartan or pharmaceutically acceptable forms thereof may, e.g., be administered in doses of about 0.01 to about 10 mg/kg of bodyweight per day, preferably is about 0.05 to about 8 mg/kg of bodyweight, even more preferably about 0.1 to about 5 mg/kg of bodyweight, even more preferably about 0.2 to about 4 mg/kg of bodyweight, even more preferably about 0.3 to about 3 mg/kg of bodyweight, even more preferably about 0.4 to about 2.5 mg/kg of bodyweight, even more preferably about 0.5 to about 2 mg/kg of bodyweight, most preferred about 0.75 to about 1.5 mg/kg of bodyweight per day, or e.g. 1.25 mg, 2.5 mg, 5 mg or 10 mg per day.

A practitioner skilled in the art is able to prepare telmisartan or pharmaceutically acceptable forms thereof for administration according to a desired dose.

### EXAMPLES

The following examples serve to further illustrate the present invention; but the same should not be construed as a limitation of the scope of the invention disclosed herein.

### Example 1 Field Study Evaluating Velagliflozin Oral Solution in Combination with Semintra® (Telmisartan) in Cats with Chronic Kidney Disease and/or Hypertension

Cats are randomized to either velagliflozin, telmisartan or the combination of velagliflozin and telmisartan. During study period, cats are evaluated regularly including a full physical exam, Systolic Blood Pressure (SBP) measurement, body weight, abdominal ultrasound, laboratory blood evaluation and urinalysis. The following parameters are assessed:
Systolic Blood Pressure (SBP)
Clinical signs (e.g., polyuria, polydipsia, food uptake, general status, dehydration, fur condition, body weight loss, reduction in body condition score (BCS), vomiting, anaemia)
Owner assessment on quality of life, veterinarians' assessment of overall control of disease.
International Renal Interest Society (IRIS) Staging
Complete blood count (CBC) and Full Serum Biochemistry including renal parameters (e.g., serum creatinine, Blood Urea Nitrogen (BUN), Symmetric dimethylarginine (SDMA), Fibroblast growth factor 23 (FGF23))
Urinalysis, urine protein to creatinine ratio (UPC), Urine Culture and if applicable Sensitivity (Urine C/S), and Urine specific gravity (USG).
number of renal events/hypertensive events, time to event.
start of further renal/antihypertensive treatment.

The results of the clinical field trial show a significant and clinically relevant delay in progression of renal disease and/or hypertension, delay of onset of hypertension in cats with renal disease, prolongation of survival time and the time to event (event is defined as renal event, e.g., hospitalization, azotemic crisis, renal death/euthanasia, target organ damage (TOD) (e.g., blindness), high blood pressure events, anaemia), a better quality of life, and a lower number of (re-)occurrence of renal/hypertensive events, of the combined treatment compared to treatment with either velagliflozin or telmisartan. Additionally, clinical parameters and renal biomarkers show a higher degree of improvement compared to single treatment.

Example 2 Exploratory Field Study Evaluating Velagliflozin Oral Solution in Combination with Semintra® (Telmisartan) for the Treatment of Dogs Diagnosed Within Chronic Kidney Disease and/or Hypertension

Dogs are randomized to either velagliflozin, telmisartan or the combination of velagliflozin and telmisartan. During study period, dogs are evaluated regularly including a full physical exam, SBP measurement, body weight, abdominal ultrasound, laboratory blood evaluation and urinalysis.

The following parameters are assessed:
Systolic Blood Pressure (SBP)
Clinical signs (e.g., polyuria, polydipsia, food uptake, general status, dehydration, fur condition, body weight loss, reduction in BCS, vomiting, anaemia)
Owner assessment on quality of life, veterinarians' assessment of overall control of disease
IRIS Staging
CBC and Full Serum Biochemistry including renal parameters (e.g., serum creatinine, BUN, SDMA, FGF23)
Urinalysis, UPC, Urine Culture and if applicable Sensitivity (Urine C/S), and USG.
number of renal events/hypertensive events, time to event.
start of further renal/antihypertensive treatment.

The results of the clinical field trial show a significant and clinically relevant delay in progression of renal disease and/or hypertension, delay of onset of hypertension in dogs with renal disease, prolongation of survival time and the time to event (event is defined as renal event, e.g., hospitalization, azotemic crisis, renal death/euthanasia, TOD (e.g., blindness), high blood pressure events, anaemia), a better quality of life, and a lower number of (re-)occurrence of renal/hypertensive events, of the combined treatment compared to treatment with either velagliflozin or telmisartan. Additionally, clinical parameters and renal biomarkers show a higher degree of improvement compared to single treatment.

### Example 3 Field Study Evaluating Velagliflozin Oral Solution in Combination with Semintra® (Telmisartan) in Cats with Chronic Kidney Disease and/or Hypertension

Cats are randomized to either telmisartan and placebo or telmisartan and velagliflozin. During study period, cats are evaluated regularly including a full physical exam, SBP measurement, body weight, abdominal ultrasound, laboratory blood evaluation and urinalysis.

The following parameters are assessed:
Systolic Blood Pressure (SBP)
Clinical signs (e.g., polyuria, polydipsia, food uptake, general status, dehydration, fur condition, body weight loss, reduction in BCS, vomiting, anaemia)
Owner assessment on quality of life, veterinarians' assessment of overall control of disease
IRIS Staging
CBC and Full Serum Biochemistry including renal parameters (e.g., serum creatinine, BUN, SDMA, FGF23)
Urinalysis, UPC, Urine Culture and if applicable Sensitivity (Urine C/S), and USG
number of renal events/hypertensive events, time to event.
start of further renal/antihypertensive treatment.

The results of the clinical field trial show a significant and clinically relevant delay in progression of renal disease and/or hypertension, delay of onset of hypertension in cats with renal disease, prolongation of survival time and the time to event (event is defined as renal event, e.g., hospitalization, azotemic crisis, renal death/euthanasia, TOD (e.g., blindness), high blood pressure events, anaemia), a better quality of life, and a lower number of (re-)occurrence of renal/hypertensive events, of the combined treatment compared to treatment with either velagliflozin or telmisartan. Additionally, clinical parameters and renal biomarkers show a higher degree of improvement compared to single treatment.

### Example 4 Exploratory Field Study Evaluating Velagliflozin Oral Solution in Combination with Semintra® (Telmisartan) for the Treatment of Dogs Diagnosed Within Chronic Kidney Disease and/or Hypertension

Dogs are randomized to either telmisartan and placebo or telmisartan and velagliflozin. During study period, dogs are evaluated regularly including a full physical exam, SBP measurement, body weight, abdominal ultrasound, laboratory blood evaluation and urinalysis.

The following parameters are assessed:
Systolic Blood Pressure (SBP)
Clinical signs (e.g., polyuria, polydipsia, food uptake, general status, dehydration, fur condition, body weight loss, reduction in BCS, vomiting, anaemia)
Owner assessment on quality of life, veterinarians' assessment of overall control of disease
IRIS Staging
CBC and Full Serum Biochemistry including renal parameters (e.g., serum creatinine, BUN, SDMA, FGF23)
Urinalysis, UPC, Urine Culture and if applicable Sensitivity (Urine C/S), and USG
number of renal events/hypertensive events, time to event
start of further renal/antihypertensive treatment.

The results of the clinical field trial show a significant and clinically relevant delay in progression of renal disease and/or hypertension, delay of onset of hypertension in dogs with renal disease, prolongation of survival time and the time to event (event is defined as renal event, e.g., hospitalization, azotemic crisis, renal death/euthanasia, TOD (e.g., blindness), high blood pressure events, anaemia), a better quality of life, and a lower number of (re-)occurrence of renal/hypertensive events, of the combined treatment compared to treatment with either velagliflozin or telmisartan. Additionally, clinical parameters and renal biomarkers show a higher degree of improvement compared to single treatment.

### REFERENCES

(1) Acierno et al., J Vet Intern Med. 2018: 32: 1803-1822
(2) Ames et al., Am J Vet Res 2015; 76:1041-50
(3) Atsuo T et al., Naunyn-Schmiedeberg's Archives of Pharmacology 2018; 391(4): 395-406
(4) Dekkers and Gansevoort, Nephrol Dial Transplant (2020) 35: 133-142
(5) Jepson RE, J Feline Med Surg. 2011;13:25-34
(6) LeBlanc et al., Journal of the American Veterinary Assoc., 2011, Vol. 238, No. 7, Pages: 915-921
(7) Shufei Z et al., Biomedicine & Pharmacotherapy 2021, XP086927259
(8) Wheeler et al., Diabetes Ther (2020) 11:2757-2774
(9) EP 3 508 222
(10) US 2015/2792977
(11) US 2020/054656
(12) US 2023/000816
(13) U.S. Pat. No. 6,358,986
(14) U.S. Pat. No. 6,410,742
(15) WO 00/43370
(16) WO 03/037876
(17) WO 2001/027128
(18) WO 2003/099836
(19) WO 2005/012326
(20) WO 2007/140191
(21) WO 2008/040774 A3
(22) WO 2008/042688
(23) WO 2010/023594
(24) WO 2015/091313 A1
(25) WO 2015/110402 A1
(26) WO 2019/059557
(27) WO 2021/092341
(28) WO 2021/105152 A1
(29) WO 2022/036506
(30) WO 2023/006745
(31) WO 2023/006747

The following clauses are also within the spirit of the present invention and are therefore part of this disclosure. It is further within the spirit of the present invention and this disclosure to further combine the subject-matter of the following clauses with any further aspects, embodiments and/or preferred embodiments as herein disclosed.
1. One or more SGLT-2 inhibitors or pharmaceutically acceptable forms thereof in combination with telmisartan or pharmaceutically acceptable forms thereof for use as a medicament.
2. The one or more SGLT-2 inhibitors or pharmaceutically acceptable forms thereof in combination with telmisartan or pharmaceutically acceptable forms thereof for use according to clause 1 in a method of prevention and/or treatment of one or more renal diseases and/or hypertension in a non-human mammal/non-human mammal patient, in particular a canine/canine patient or a feline/feline patient.
3. The one or more SGLT-2 inhibitors or pharmaceutically acceptable forms thereof in combination with telmisartan or pharmaceutically acceptable forms thereof for use according to clause 2 in a method of treatment of one or more renal diseases and/or hypertension in a non-human mammal/non-human mammal patient, in particular a canine/canine patient or a feline/feline patient.
4. The one or more SGLT-2 inhibitors or pharmaceutically acceptable forms thereof in combination with telmisartan or pharmaceutically acceptable forms thereof for use according to clause 2 or 3, wherein the one or more renal diseases are selected from the group consisting of: renal dysplasia, glomerulopathy, polycystic kidney disease, amyloidosis, tubulo-nephritis/tubulointerstitial nephritis (TIN), acute kidney disease, chronic kidney disease, proteinuria.
5. The one or more SGLT-2 inhibitors or pharmaceutically acceptable forms thereof in combination with telmisartan or pharmaceutically acceptable forms thereof for use according to clause 4, wherein the one or more renal diseases are selected from the group consisting of: acute kidney disease, chronic kidney disease; and wherein the non-human mammal/non-human mammal patient is a feline/feline patient; preferably a feline patient in need of such prevention and/or treatment; more preferably a cat in need of such prevention and/or treatment.
6. The one or more SGLT-2 inhibitors or pharmaceutically acceptable forms thereof in combination with telmisartan or pharmaceutically acceptable forms thereof for use according to clause 2 or 3, wherein the hypertension is selected from the group consisting of: systemic hypertension, glomerular hypertension, situational hypertension, secondary hypertension and idiopathic hypertension.
7. The one or more SGLT-2 inhibitors or pharmaceutically acceptable forms thereof in combination with telmisartan or pharmaceutically acceptable forms thereof for use according to clause 6, wherein the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride.
8. The one or more SGLT-2 inhibitors or pharmaceutically acceptable forms thereof in combination with telmisartan or pharmaceutically acceptable forms thereof for use according to any one of clauses 1 to 7, wherein the one or more SGLT-2 inhibitors are selected from the group consisting of:
   (1) a glucopyranosyl-substituted benzene derivative of the formula (1)

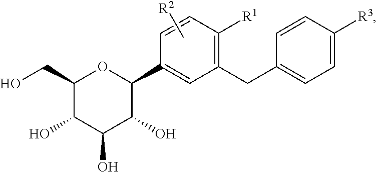

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxylethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R3 is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
   (2) Velagliflozin, represented by formula (2):

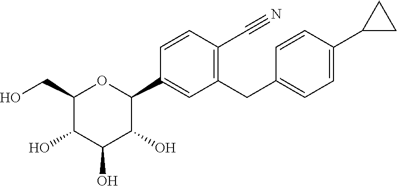 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

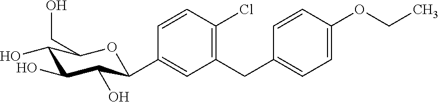

(4) Canagliflozin, represented by formula (4):

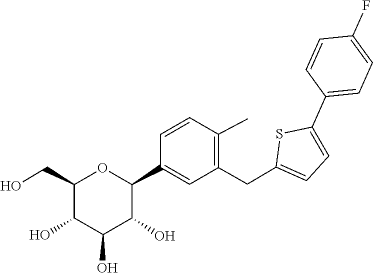 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

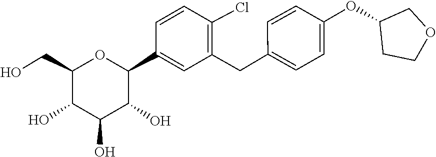 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

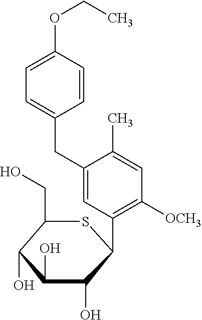

(7) Tofogliflozin, represented by formula (7):

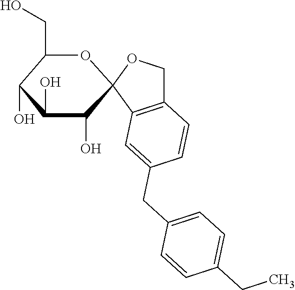

(8) Ipragliflozin, represented by formula (8):

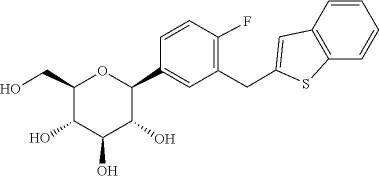 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

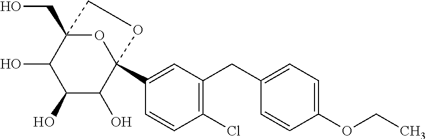 [SMILES: CCOc1ccc(Cc2cc([C@]34OC[C@](CO)(O3)C(O)[C@H](O)C4O)ccc2Cl)cc1]

(10) Atigliflozin, represented by formula (10):

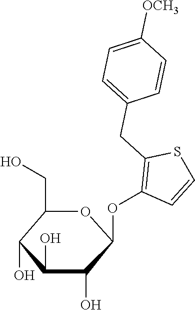

(11) Remogliflozin, represented by formula (11):

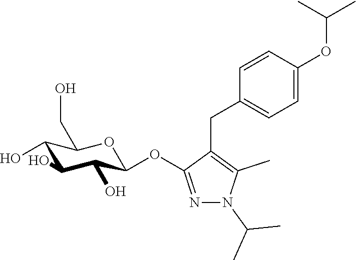

(11A) Remogliflozin etabonate, represented by formula (11A):

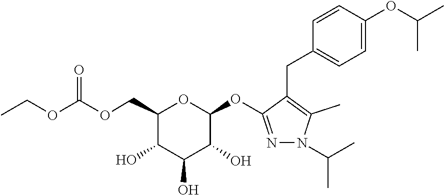 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

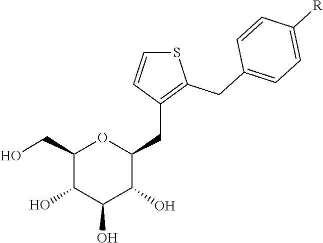

wherein R denotes methoxy or trifluoromethoxy;
   (13) 1-(β-D-giucopyranosyl)-4-methyl-3-[5-(4-fluorophenyl)-2-thienyimethyl]benzene, represented by formula (13);

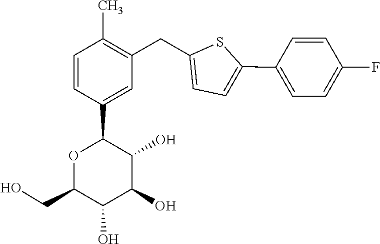 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

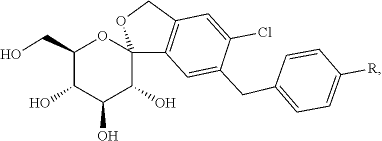

wherein R denotes methoxy, trifluoromethoxy, ethoxy, ethyl, isopropyl or tert. butyl;
   (15) a pyrazole-O-glucoside derivative of the formula (15)

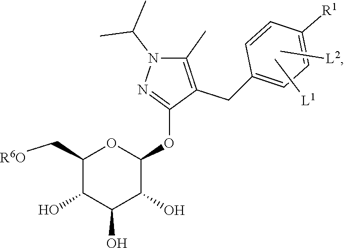

wherein
R¹ denotes C₁₋₃-alkoxy,
L¹, L² independently of each other denote H or F,
R⁶ denotes H, (C₁₋₃-alkyl) carbonyl, (C₁₋₆-alkyl) oxycarbonyl, phenyloxycarbonyl, benzyloxycarbonyl or benzylcarbonyl;
   (16) Sotagliflozin, represented by formula (16):

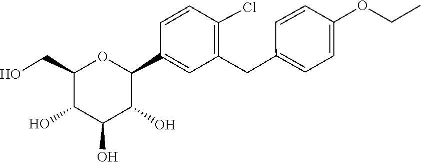 [SMILES: CCOc1ccc(Cc2cc([C@@H]3O[C@H](CO)[C@@H](O)[C@H](O)[C@H]3O)ccc2Cl)cc1]

(17) Sergliflozin, represented by formula (17):

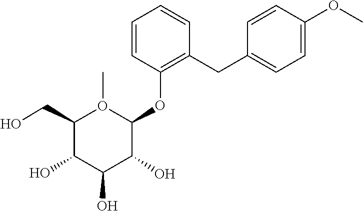

(18) a compound represented by formula (18):

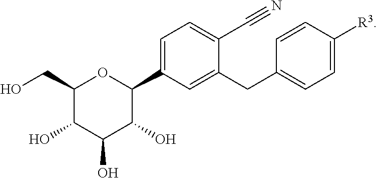

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl) carbonyl, (C₁₋₁₈-alkyl) oxycarbonyl, phenylcarbonyl and phenyl-(C₁₋₃-alkyl)-carbonyl;
   (19) Bexagliflozin, represented by formula (19):

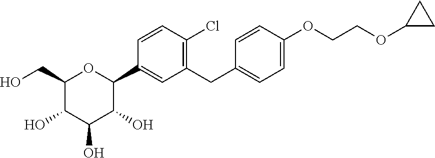 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

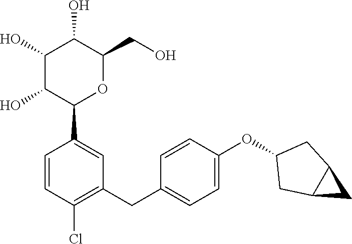 [SMILES: OC[C@H]1O[C@@H](c2ccc(Cl)c(Cc3ccc(O[C@H]4C[C@@H]5C[C@@H]5C4)cc3)c2)[C@H](O)[C@H](O)[C@@H]1O]

(21) Rongliflozin, represented by formula (21):

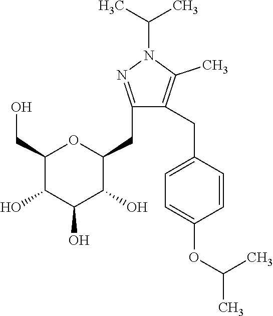 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
   (23) Enavogliflozin, represented by formula (23):

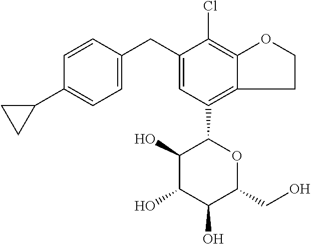 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

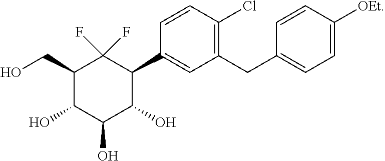 [SMILES: CCOc1ccc(Cc2cc([C@H]3[C@H](O)[C@@H](O)[C@H](O)[C@@H](CO)C3(F)F)ccc2Cl)cc1]

9. The one or more SGLT-2 inhibitors or pharmaceutically acceptable forms thereof in combination with telmisartan or pharmaceutically acceptable forms thereof for use according to any one of clauses 1 to 8, wherein the pharmaceutically acceptable form thereof is a crystalline complex between the one or more SGLT-2 inhibitors and one or more amino acids, preferably proline, more preferably L-proline; and most preferably is co-crystal of the one or more SGLT2 inhibitors, L-proline and crystalline water.
10. The one or more SGLT-2 inhibitors or pharmaceutically acceptable forms thereof in combination with telmisartan or pharmaceutically acceptable forms thereof for use according to any one of clauses 1 to 9, wherein velagliflozin or pharmaceutically acceptable forms thereof is to be administered as single SGLT-2 inhibitor in combination with telmisartan or pharmaceutically acceptable forms thereof.
11. The one or more SGLT-2 inhibitors or pharmaceutically acceptable forms thereof in combination with telmisartan or pharmaceutically acceptable forms thereof for use according to any one of clauses 1 to 10, wherein velagliflozin or pharmaceutically acceptable forms thereof is used as single SGLT-2 inhibitor, and wherein the use is in a method of prevention and/or treatment of CKD or hypertension in a canine/canine patient.
12. The one or more SGLT-2 inhibitors or pharmaceutically acceptable forms thereof in combination with telmisartan or pharmaceutically acceptable forms thereof for use according to any one of clauses 1 to 10, wherein velagliflozin or pharmaceutically acceptable forms thereof is used as single SGLT-2 inhibitor, and wherein the use is in a method of prevention and/or treatment of CKD or hypertension in a feline/feline patient.
13. The one or more SGLT-2 inhibitors or pharmaceutically acceptable forms thereof in combination with telmisartan or pharmaceutically acceptable forms thereof for use according to any one of clauses 1 to 10, wherein velagliflozin or pharmaceutically acceptable forms thereof is used as single SGLT-2 inhibitor, and wherein the use is in a method of treatment of CKD or hypertension in a canine/canine patient.
14. The one or more SGLT-2 inhibitors or pharmaceutically acceptable forms thereof in combination with telmisartan or pharmaceutically acceptable forms thereof for use according to any one of clauses 1 to 10, wherein velagliflozin or pharmaceutically acceptable forms thereof is used as single SGLT-2 inhibitor, and wherein the use is in a method of treatment of CKD or hypertension in a feline/feline patient.
15. The one or more SGLT-2 inhibitors or pharmaceutically acceptable forms thereof in combination with telmisartan or pharmaceutically acceptable forms thereof for use according to any one of clauses 1 to 14, wherein the one or more SGLT-2 inhibitors or pharmaceutically acceptable forms thereof are administered orally, parenterally, intravenously, subcutaneously or intramuscularly, preferably orally.
16. The one or more SGLT-2 inhibitors or pharmaceutically acceptable forms thereof in combination with telmisartan or pharmaceutically acceptable forms thereof for use according to any one of clauses 1 to 15, wherein the one or more SGLT-2 inhibitors or pharmaceutically acceptable forms thereof are to be administered at a dose of 0.01 mg/kg bodyweight to 10 mg/kg bodyweight per day, preferably at a dose of 0.01 mg/kg bodyweight to 5 mg/kg bodyweight per day, more preferably at a dose of 0.01 mg/kg bodyweight to 4 mg/kg bodyweight per day, even more preferably at a dose of 0.01 mg/kg bodyweight to 3 mg/kg bodyweight per day, even more preferably at a dose of 0.01 mg/kg bodyweight to 2 mg/kg bodyweight per day, even more preferably at a dose of 0.01 mg/kg bodyweight to 1 mg/kg bodyweight per day, even more preferably at a dose of 0.01 mg/kg bodyweight to 0.5 mg/kg bodyweight per day, most preferably at a dose of 0.01 mg/kg bodyweight to 0.3 mg/kg bodyweight per day.
17. The one or more SGLT-2 inhibitors or pharmaceutically acceptable forms thereof in combination with telmisartan or pharmaceutically acceptable forms thereof for use according to any one of clauses 1 to 16, wherein such one or more SGLT-2 inhibitors or pharmaceutically acceptable forms thereof is to be administered once per day or twice per day, preferably once per day
18. The one or more SGLT-2 inhibitors or pharmaceutically acceptable forms thereof in combination with telmisartan or pharmaceutically acceptable forms thereof for use according to any one of clauses 1 to 17, wherein telmisartan or pharmaceutically acceptable forms thereof is to be administered at a dose of about 0.01 to about 10 mg/kg of bodyweight per day, preferably is about 0.05 to about 8 mg/kg of bodyweight, even more preferably about 0.1 to about 5 mg/kg of bodyweight, even more preferably about 0.2 to about 4 mg/kg of bodyweight, even more preferably about 0.3 to about 3 mg/kg of bodyweight, even more preferably about 0.4 to about 2.5 mg/kg of bodyweight, even more preferably about 0.5 to about 2 mg/kg of bodyweight, most preferred about 0.75 to about 1.5 mg/kg of bodyweight per day.
19. The one or more SGLT-2 inhibitors or pharmaceutically acceptable forms thereof in combination with telmisartan or pharmaceutically acceptable forms thereof for use according to any one of clauses 1 to 18, wherein telmisartan or pharmaceutically acceptable forms thereof is to be administered once per day or twice per day, preferably once per day.
20. The one or more SGLT-2 inhibitors or pharmaceutically acceptable forms thereof in combination with telmisartan or pharmaceutically acceptable forms thereof for use according to any one of clauses 1 to 19, wherein the one or more SGLT-2 inhibitors or pharmaceutically acceptable forms thereof are administered in combination with telmisartan or pharmaceutically acceptable forms thereof, preferably wherein the one or more SGLT-2 inhibitors or pharmaceutically acceptable forms thereof is to be administered before, after or concomitantly with administering telmisartan or pharmaceutically acceptable forms thereof.
21. The one or more SGLT-2 inhibitors or pharmaceutically acceptable forms thereof in combination with telmisartan or pharmaceutically acceptable forms thereof for use according to any one of clauses 1 to 20, wherein the preventive and/or therapeutic effect is characterized by one or more of the following clinical and/or biochemical parameters:
   improved renal efficiency, characterized by a reduction of proteinuria-as well as a reduction and/or stabilization of serum SDMA and/or serum creatinine and/or FGF23 and/or blood urea nitrogen (BUN) and/or hyperphosphataemia;
   increase of the production of ketone bodies in the liver, characterized by increased plasma levels of 3-hydroxybutyric acid and/or the corresponding acylcarnitines i.e., hydroxybutyrylcarnitine and increased plasma levels of one or more of the branched-chain amino acids (e.g., valine, leucine and isoleucine);
   delayed onset of hypertension and/or prevention of target organ damage and/or improved blood pressure;
   improved hydration status;
   delayed onset of renal failure, preferably at least by 1, 2, 3, 4, 5, 6, 7, 8, 9, 10, 11, 12 or more months, or delayed and/or stopped progression of the one or more renal diseases, in particular chronic kidney disease, and/or improvement of the classification stage of the one or more renal diseases, in particular CKD (e.g., from stage III to stage II);
   promoting diuresis in order to reduce renal failure with oliguria and/or hypertension;
   longer survival time of the non-human mammal patient, preferably at least by 1, 2, 3, 4, 5, 6, 7, 8, 9, 10, 11, 12 or more months and/or lower renal related mortality and/or morbidity;
   improved clinical signs, such as reduced polydipsia, polyuria, vomiting and/or lethargy;
   higher quality of life.
22. The one or more SGLT-2 inhibitors or pharmaceutically acceptable forms thereof in combination with telmisartan or pharmaceutically acceptable forms thereof for use according to any one of clauses 1 to 21, wherein the systolic blood pressure (SBP) value measured for the non-human mammal/non-human mammal patient, in particular a canine/canine patient or a feline/feline patient, in need thereof is decreased after the period of the treatment by at least 5 mmHg, preferably by at least 10 mmHg, more preferably by at least 20 mmHg, in particular by 5 to 100 mmHg, more preferably 5 to 50 mmHg, most preferably by 10 to 50 mmHg, in relation to the baseline SBP value measured for the non-human mammal/non-human mammal patient, in particular a canine/canine patient or a feline/feline patient, more preferably a cat or a dog, prior to the period of treatment.
23. The one or more SGLT-2 inhibitors or pharmaceutically acceptable forms thereof in combination with telmisartan or pharmaceutically acceptable forms thereof for use according to any one of clauses 1 to 22, wherein the method comprises measurement of the SBP and optionally identification of TOD followed by administration of a therapeutically effective amount of the one or more SGLT-2 inhibitors or pharmaceutically acceptable forms thereof to the non-human mammal/non-human mammal patient, in particular a canine/canine patient or a feline/feline patient, wherein the therapeutically effective amount of the one or more SGLT-2 inhibitors or pharmaceutically acceptable forms thereof is administered in a daily dosage amount that may be varied over a treatment period depending on subsequent measurements of the SBP.
24. The one or more SGLT-2 inhibitors or pharmaceutically acceptable forms thereof in combination with telmisartan or pharmaceutically acceptable forms thereof for use according to any one of clauses 1 to 23, wherein the hypertension is non-refractory to the treatment with ACE inhibitors in the non-human mammal/non-human mammal patient, in particular a canine/canine patient or a feline/feline patient, in particular the cat or dog to be treated, most preferably the dog to be treated.
25. A pharmaceutical composition comprising one or more SGLT2 inhibitors or pharmaceutically acceptable forms thereof in combination with telmisartan or pharmaceutically acceptable forms thereof for use as defined in any one of clauses 1 to 24, wherein preferably such pharmaceutical composition is a fixed-dose-combination (FDC) of the one or more SGLT-2 inhibitors or pharmaceutically acceptable forms thereof and telmisartan or a pharmaceutically acceptable from thereof, wherein more preferably such FDC is a solid or a liquid formulation.

1. A method of prevention and/or treatment of one or more renal diseases and/or hypertension in a non-human mammal comprising administering to the non-human mammal a pharmaceutical composition comprising one or more SGLT-2 inhibitors or pharmaceutically acceptable forms thereof in combination with telmisartan or a pharmaceutically acceptable form thereof.

2. The method according to claim 1, wherein the non-human mammal is a canine or a feline, in particular.

3. (canceled)

4. The method according to claim 1, wherein the one or more renal diseases is selected from the group consisting of renal dysplasia, glomerulopathy, polycystic kidney disease, amyloidosis, tubulo-nephritis/tubulointerstitial nephritis (TIN), acute kidney disease, chronic kidney disease, and proteinuria.

5. The method according to claim 4, wherein the one or more renal diseases are is selected from the group consisting of acute kidney disease, and chronic kidney disease, and the non-human mammal is as feline.

6. The method of claim 1, wherein the method comprises prevention and/or treatment of hypertension selected from the group consisting of systemic hypertension, glomerular hypertension, situational hypertension, secondary hypertension, and idiopathic hypertension.

7. The method according to claim 6, wherein the method comprises prevention and/or treatment of secondary hypertension selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, by glucocorticoids, mineralocorticoids, erythropoiesis-stimulating agents, ephedrine and/or high dose sodium chloride.

8. The one or more SGLT-2 inhibitors or pharmaceutically acceptable forms thereof in combination with telmisartan or pharmaceutically acceptable forms thereof for use according to claim 1, wherein the one or more SGLT-2 inhibitors are selected from the group consisting of:
(1) a glucopyranosyl-substituted benzene derivative of the formula (1) 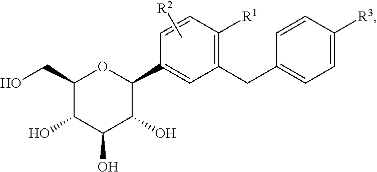
wherein R¹ denotes cyano, Cl or methyl;
R² denotes H, methyl, methoxy or hydroxy; and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2): 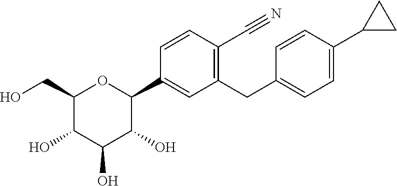 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by formula (3): 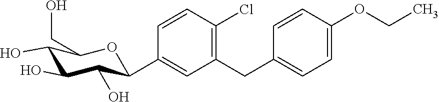
(4) Canagliflozin, represented by formula (4): 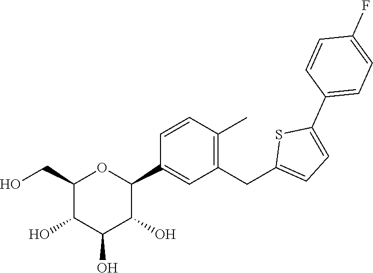 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by formula (5): 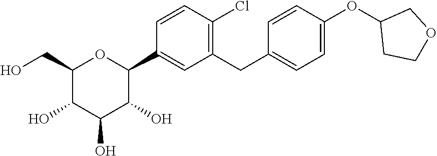 [SMILES: OC[C@H]1O[C@@H](c2ccc(Cl)c(Cc3ccc(OC4CCOC4)cc3)c2)[C@H](O)[C@@H](O)[C@@H]1O]
(6) Luseogliflozin, represented by formula (6): 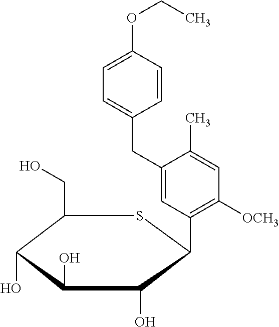
(7) Tofogliflozin, represented by formula (7): 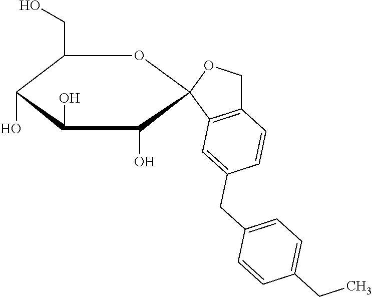
(8) Ipragliflozin, represented by formula (8): 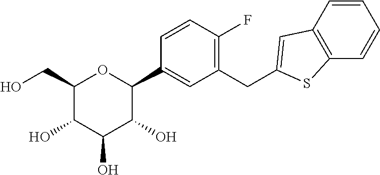 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by formula (9): 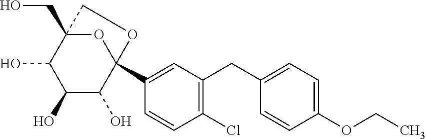 [SMILES: CCOc1ccc(Cc2cc([C@]34OC[C@](CO)(O3)[C@@H](O)[C@H](O)[C@H]4O)ccc2Cl)cc1]
(10) Atigliflozin, represented by formula (10): 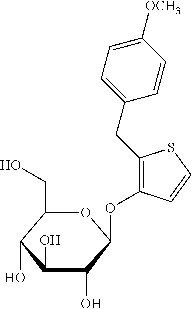
(11) Remogliflozin, represented by formula (11): 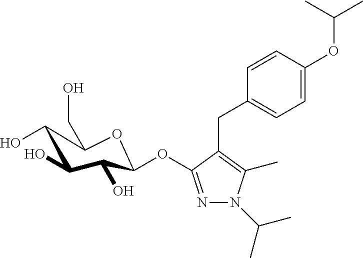
(11A) Remogliflozin etabonate, represented by formula (11A): 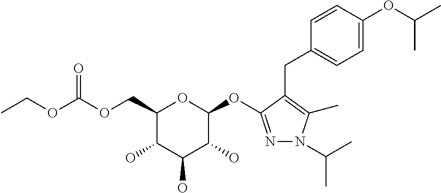 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative of the formula (12) 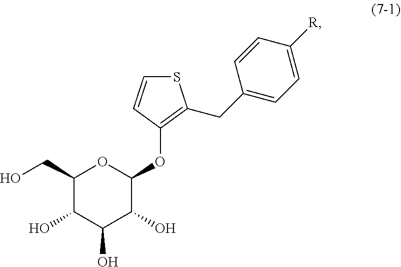
wherein R denotes methoxy or trifluoromethoxy;
(13) 1-(β-D-glucopyranosyl)-4-methyl-3-[5-(4-fluorophenyl)-2-thienylmethyl]benzene, represented by formula (13); 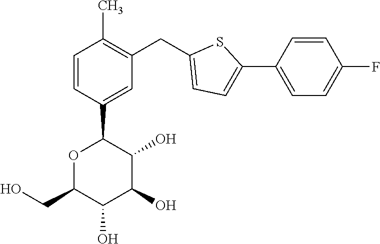 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative of the formula (14): 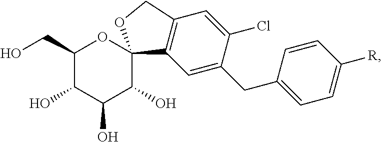
wherein R denotes methoxy, trifluoromethoxy, ethoxy, ethyl, isopropyl or tert. butyl;
(15) a pyrazole-O-glucoside derivative of the formula (15) 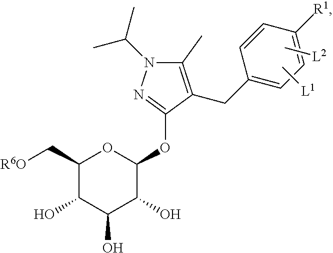
wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16): 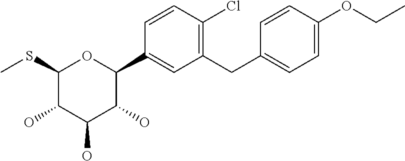 [SMILES: CCOc1ccc(Cc2cc([C@@H]3O[C@H](SC)[C@@H](O)[C@H](O)[C@H]3O)ccc2Cl)cc1]
(17) Sergliflozin, represented by formula (17): 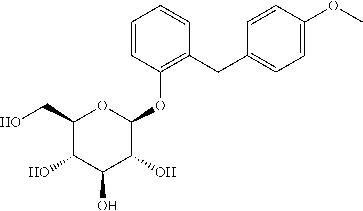 [SMILES: COc1ccc(Cc2ccccc2O[C@@H]2O[C@H](CO)[C@@H](O)[C@H](O)[C@H]2O)cc1]
(18) a compound represented by formula (18): 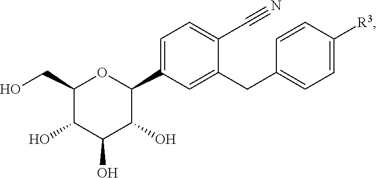
wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano,
or a derivative thereof wherein one or more hydroxyl groups of the β-D-glucopyranosyl group are acylated with groups selected from (C₁₋₁₈-alkyl)carbonyl, (C₁₋₁₈-alkyl) oxycarbonyl, phenylcarbonyl and phenyl-(C₁₋₃-alkyl)-carbonyl;
(19) Bexagliflozin, represented by formula (19): 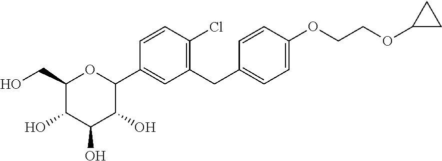 [SMILES: OC[C@H]1OC(c2ccc(Cl)c(Cc3ccc(OCCOC4CC4)cc3)c2)[C@H](O)[C@@H](O)[C@@H]1O]
(20) Janagliflozin, represented by formula (20): 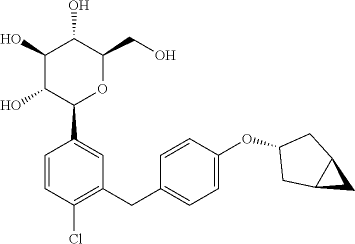 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin, represented by formula (21): 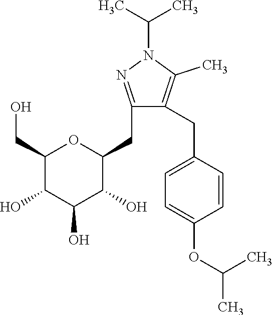 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin;
(23) Enavogliflozin, represented by formula (23): 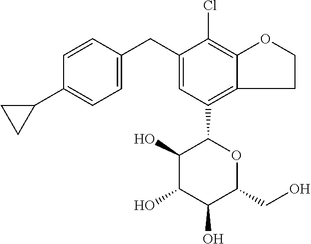 [SMILES: OC[C@H]1O[C@@H](c2cc(Cc3ccc(C4CC4)cc3)c(Cl)c3c2CCO3)[C@H](O)[C@@H](O)[C@@H]1O]
and
(24) TFC-039, represented by formula (24):

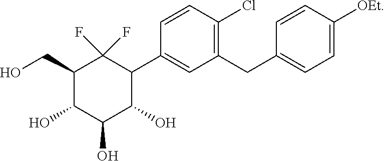 [SMILES: CCOc1ccc(Cc2cc(C3[C@H](O)[C@@H](O)[C@H](O)[C@@H](CO)C3(F)F)ccc2Cl)cc1]

9. The method according to claim 1, wherein the pharmaceutically acceptable form thereof is a crystalline complex between the one or more SGLT-2 inhibitors and one or more amino acids, such as proline, L-proline; or a co-crystal of the one or more SGLT2 inhibitors, L-proline and crystalline water.

10. The method according to claim 1, wherein the one or more SGLT-2 inhibitors comprises velagliflozin.

11. The method according to claim 10, wherein the method comprises prevention and/or treatment of CKD or hypertension in a canine.

12. The method according to claim 1, wherein the method comprises prevention and/or treatment of CKD or hypertension in a feline.

13. The method according to claim 1, wherein the one or more SGLT-2 inhibitors comprises velagliflozin, and the method comprises treatment and/or prevention of CKD or hypertension in a canine.

14. The method according to claim 1, wherein the one or more SGLT-2 inhibitors comprises velagliflozin, and the method comprises treatment and/or prevention of CKD or hypertension in a feline.

15. The method according to claim 1, wherein the one or more SGLT-2 inhibitors or pharmaceutically acceptable forms thereof is administered orally, parenterally, intravenously, subcutaneously or intramuscularly.

16. The method according to claim 1, wherein the one or more SGLT-2 inhibitors or pharmaceutically acceptable forms thereof is administered at a dose of 0.01 mg/kg bodyweight to 10 mg/kg bodyweight per day, or at a dose of 0.01 mg/kg bodyweight to 5 mg/kg bodyweight per day, or at a dose of 0.01 mg/kg bodyweight to 4 mg/kg bodyweight per day, or at a dose of 0.01 mg/kg bodyweight to 3 mg/kg bodyweight per day, or at a dose of 0.01 mg/kg bodyweight to 2 mg/kg bodyweight per day, or at a dose of 0.01 mg/kg bodyweight to 1 mg/kg bodyweight per day, or at a dose of 0.01 mg/kg bodyweight to 0.5 mg/kg bodyweight per day, or at a dose of 0.01 mg/kg bodyweight to 0.3 mg/kg bodyweight per day.

17. The method according to claim 1, wherein the one or more SGLT-2 inhibitors or pharmaceutically acceptable forms thereof is administered once per day or twice per day.

18. The method according to claim 1, wherein telmisartan or a pharmaceutically acceptable form thereof is administered at a dose of about 0.01 to about 10 mg/kg of bodyweight per day, or about 0.05 to about 8 mg/kg of bodyweight, or about 0.1 to about 5 mg/kg of bodyweight, or about 0.2 to about 4 mg/kg of bodyweight, or about 0.3 to about 3 mg/kg of bodyweight, or about 0.4 to about 2.5 mg/kg of bodyweight, or about 0.5 to about 2 mg/kg of bodyweight, or 0.75 to about 1.5 mg/kg of bodyweight per day.

19. The method according to claim 1, wherein telmisartan or a pharmaceutically acceptable form thereof is to be administered once per day or twice per day.

20. The method according to claim 1, wherein the one or more SGLT-2 inhibitors or pharmaceutically acceptable forms thereof is administered before, after or concomitantly with administering telmisartan or a pharmaceutically acceptable form thereof.

21. The method according to claim 1, wherein the preventive and/or therapeutic effect is characterized by one or more of the following clinical and/or biochemical parameters:
improved renal efficiency, characterized by a reduction of proteinuria and/or a reduction and/or stabilization of blood parameters, such as serum SDMA, serum creatinine, FGF23, blood urea nitrogen (BUN) and/or hyperphosphatemia, and/or slowed reduction in glomerular filtration rate (GFR);
increase of the production of ketone bodies in the liver, characterized by increased plasma levels of 3-hydroxybutyric acid and/or the corresponding acylcarnitines i.e., hydroxybutyrylcarnitine and increased plasma levels of one or more of the branched-chain amino acids (e.g., valine, leucine and isoleucine);
delayed onset of hypertension and/or prevention of target organ damage and/or improved blood pressure;
improved hydration status;
delayed onset of renal failure, by at least one month to 12 or more months, or delayed and/or stopped progression of the one or more renal diseases, such as chronic kidney disease, and/or improvement of the classification stage of the one or more renal diseases, such as CKD (e.g., from stage III to stage II);
promoting diuresis in order to reduce renal failure with oliguria and/or hypertension;
longer survival time of the non-human mammal patient, by at least one month to 12 or more months and/or lower renal related mortality and/or morbidity;
improved clinical signs, such as reduced polydipsia, polyuria, vomiting, lethargy, weight loss, and/or signs associated with dehydration; and
higher quality of life.

22. The method according to claim 1, wherein the systolic blood pressure (SBP) value measured for the non-human mammal is decreased after the period of the treatment by at least 5 mmHg, or by at least 10 mmHg, or by at least 20 mmHg, or by 5 to 100 mmHg, or 5 to 50 mmHg, or by 10 to 50 mmHg, in relation to a baseline SBP value measured for the non-human mammal.

23. The method according to claim 1, further comprising measuring the systolic blood pressure (SBP) and optionally identification of target organ damage (TOD) followed by administration of a therapeutically effective amount of the one or more SGLT-2 inhibitors or pharmaceutically acceptable forms thereof to the non-human mammal, wherein the therapeutically effective amount of the one or more SGLT-2 inhibitors or pharmaceutically acceptable forms thereof is administered in a daily dosage amount that is varied over a treatment period in response to subsequent measurements of the SBP.

24. The method according to claim 1, wherein the method comprises prevention and/or treatment of hypertension that is non-refractory to the treatment with ACE inhibitors in the non-human mammal.

25. A pharmaceutical composition comprising one or more SGLT2 inhibitors or pharmaceutically acceptable forms thereof in combination with telmisartan or a pharmaceutically acceptable form thereof, wherein the pharmaceutical composition is a fixed-dose-combination (FDC) of the one or more SGLT-2 inhibitors or pharmaceutically acceptable forms thereof and telmisartan or a pharmaceutically acceptable from thereof, and the FDC is a solid or a liquid formulation.
